(19) United States
(12) Patent Application Publication
(10) Pub. No.: US 2013/0172731 A1
(43) Pub. Date: Jul. 4, 2013

(54) IMAGE-OVERLAY MEDICAL EVALUATION DEVICES AND TECHNIQUES

(71) Applicant: Philip D. Gole, Grand Rapids, MI (US)

(72) Inventor: Philip D. Gole, Grand Rapids, MI (US)

(21) Appl. No.: 13/731,144

(22) Filed: Dec. 31, 2012

Related U.S. Application Data
(60) Provisional application No. 61/582,007, filed on Dec. 30, 2011.
(60) Provisional application No. 61/597,494, filed on Feb. 10, 2012.

(51) Int. Cl. A61B 5/00

(52) U.S. Cl. CPC A61B 5/0035
USPC 600/424

(57) ABSTRACT

A system and methods are provided for evaluating the position of surgical implants or openings formed in anatomical tissue during medical procedures, in which an initial 3-dimensional image of the anatomical tissue is combinable with one or more subsequent 3-dimensional images of the same or correlating tissue, and without the use of ionizing radiation on a patient for at least the subsequent images. The system includes a software program, a computer with display, a radiographic image scanning device, and a non-radiographic image scanning device. The computer accesses a medical patient information database and a medical implant database for images used by the software program. A pre-operative image is combined with subsequent images to facilitate evaluation of the proposed placement of a surgical implant or opening relative to tissues of the patient anatomy. Methods of evaluating the accuracy of surgical guides and of fabricating surgical guides are also disclosed.

### CROSS REFERENCE TO RELATED APPLICATIONS

The present application claims the priority benefit of U.S. provisional application Ser. No. 61/582,007, filed Dec. 30, 2011, and of U.S. provisional application Ser. No. 61/597,494, filed Feb. 10, 2012, which are hereby incorporated herein by reference in their entireties.

### FIELD OF THE INVENTION

The present invention relates to medical imaging systems and methods for use in visually confirming the location or proposed location of prostheses or implants to body tissues, or for modifying body tissues such as osteotomies, osteoplasty, or modifications to softer tissues, and for the manufacturing and evaluation of surgical guides used in modifying body tissues including for attaching prostheses or implants.

### BACKGROUND OF THE INVENTION

Typical methods for attaching prostheses or implants (such as dental implants, for example) to body tissues (such as bone) involve multiple radiographic scans of at least a portion of the body with different forms of potentially harmful radiation. Such radiographic scans may be performed using X-ray, computed tomography (“CT”) scanning, cone beam computed tomography (“CBCT”), or the like, all of which use ionizing radiation to acquire diagnostic images. The scans are typically performed prior to a surgical procedure, such as to evaluate pre-operative internal anatomy, and may be performed again during and/or after the surgical procedure, such as to evaluate the positioning of prostheses, implants, tools, etc. during and/or after surgery, such as to help ensure that the desired effect is achieved. However, repeated scans expose medical patients and medical personnel to repeated doses of radiation. It also can be prohibitively costly and time-consuming to use non-ionizing radiation, such as magnetic resonance imaging (“MRI”), for such evaluations.

The desire for successful and predictable surgical results has led to significant advancement in dentistry and medicine in recent years. For example, accurate placement and retention of dental implants has significantly improved with the introduction of cone beam computed tomography (CBCT). Studies have demonstrated that CBCT technology can provide benefits of increased accuracy and lower radiation exposure compared to other radiographic scanning technologies. Additionally, the advent of CBCT in dentistry has led to the development of more precise surgical guides for use during dental implant placement.

In accordance with earlier techniques of surgical guide fabrication in the dental environment, “bench-top surgical guides” have been made by a clinician or laboratory personnel based on a diagnostic wax-up of the patient anatomy, simply by marking on a diagnostic cast of an anatomical portion of the patient. Although such methods typically provide improved accuracy over medical procedures (e.g., drilling osteotomies) free-hand or without the use of a guide, such bench-top surgical guides can still be inaccurate and unpredictable.

Known techniques of fabricating more accurate surgical guides may begin with fabricating an accurate model of a patient's dentition, such as by making an impression and pouring a cast, or using rapid prototyping techniques such as stereolithography, which can be used to produce a model directly from CT, CBCT, MRI, or laser scan data or the like. Some commercial systems for producing such CT-guided surgical guides use various radiopaque markers, navigation software, and imaging processes. However, typical commercial methods of surgical guide fabrication can be tedious and expensive for the practicing clinician, and can take days or weeks to complete, especially when some of the steps are completed at an off-site location and require shipping of casts, surgical guides, and the like. Moreover, even after a clinician uses a surgical guide to place one or more dental implants, confirming the accuracy of placement has typically required exposing the patient to a post-surgical X-ray or CBCT or CT scan, resulting in additional radiation exposure to the patient.

It is also known to take several mid-surgical periapical (i.e., around the apex of the root of a tooth) X-rays to assess surgical drill angulations and implant location during the surgical phase of implant placement. While conventional radiographic methods can determine depth and mesial-distal dimensions, a true 3-dimensional assessment is difficult to achieve using known methods. Using known methods, a patient would typically undergo an additional CBCT or CT scan to evaluate the mid-surgical or final position of an implant. However, the access to a CBCT or CT scan during surgery, additional radiation exposure, and cost to perform these procedures can be prohibitive.

### SUMMARY OF THE INVENTION

The image-overlay techniques and related systems of the present invention provide for simple, expedient, and reliable surgical guide fabrication and evaluation, which is sufficiently low in cost and short in time for its use to be justified in most cases where a surgical implant is desired. Desirable characteristics for surgical guide include precision, low cost, easy fabrication by substantially any clinician, use as a diagnostic adjunct, and facilitating the reduction of radiation (particularly ionizing radiation) exposure to the patient. The systems and techniques of the present invention facilitate the pre-operative, mid-operative, and post-operative evaluation of proposed, ongoing, and completed medical procedures, as well as the fabrication of precise surgical guides for routine use, and in substantially all cases in which surgical implants are desired, to help ensure favorable treatment outcomes for medical patients. Moreover, the techniques of the present invention generally do not require any changes to the actual surgical or other medical procedures that are used on the patient, and can be used at substantially any stage of a surgical procedure and while using standard surgical equipment.

The image-overlay techniques and systems of the present invention have the ability to achieve these benefits, including the ability quickly produce an accurate surgical guide, and to “CT-confirm” the accuracy of such guides. With access to a pre-surgical CT image and the means to create a digital image of a working model, substantially any appropriately equipped dentist or laboratory technician can create a “CT-confirmed” surgical guide, sometimes within a matter of hours. The combination of a “CT-confirmed” surgical guide and the related image-overlay techniques of the present invention reduce exposure of patients to mid-surgery and post-surgery radiation. The techniques may also be used to evaluate the actual location of an osteotomy or surgical implant within patient tissue, without the use of ionizing radiation other than an initial pre-surgical scan. The use of these techniques can have the immediate impact of reducing the radiation exposure to a patient by at least 50% during a given surgical procedure.

According to one form of the present invention, a system is provided for collecting and displaying medical images. The system includes a software program, a computer and display, a radiographic image scanning device, and a non-radiographic image scanning device. In addition, a medical patient information database and a medical implant database are accessible by the computer to provide access to images used by the software program. The software program is configured to enable the manipulation and overlaying a plurality of digital images, and the computer is configured to execute the software program. The display is in communication with the computer to display medical images. The radiographic image scanning device and the non-radiographic scanning device are both in communication with the computer. The medical patient information database stores patient medical images that are generated by the radiographic image scanning device and the non-radiographic image scanning device, for a given patient. The medical implant database stores dimensional and/or geometrical and/or 3-dimensional images for one or more medical implants. The software program is executable by the computer to overlay and align a plurality of 3-dimensional images at the display. These 3-dimensional images include (i) a first 3-dimensional image of an anatomical portion of a patient that has been collected by the radiographic image scanning device, (ii) a second 3-dimensional image of the anatomical portion of the patient that has been collected by the non-radiographic image scanning device, and (iii) a 3-dimensional image of a medical implant that has been obtained from the medical implant database and/or from either of the radiographic image scanning device and the non-radiographic image scanning device.

In one aspect, the system further includes a rapid prototyping machine in communication with the computer. The rapid prototyping machine is operable to create 3-dimensional physical models, such as of an anatomical portion of a patient, based on image data received from the computer.

In another aspect, the radiographic image scanning device is any of an X-ray device, a CT scanning device, a CBCT scanning device, and an MRI scanning device. Optionally, the non-radiographic image scanning device is an optical laser scanner.

In yet another aspect, the software program is operable to obtain the 3-dimensional image of the medical implant directly from any of (i) the radiographic image scanning device, (ii) the non-radiographic image scanning device, and (iii) the medical implant database.

In still another aspect, the non-radiographic image scanning device is configured to generate the second 3-dimensional image from either the anatomical portion of the patient, or from a physical model of the anatomical portion of the patient.

In a further aspect, the software program is configured to individually scale the size of one or more of the various 3-dimensional images at the display, so that each of the 3-dimensional images can be viewed substantially simultaneously on the display at the same size (i.e. 1:1 scale) as the other 3-dimensional images shown on the display.

According to another form of the present invention, a method is provided for evaluating the position of an opening, such as an osteotomy, formed in body tissue. The method includes the steps of scanning an anatomical portion of a patient to produce an initial 3-dimensional image. The initial 3-dimensional image includes a depiction of both internal tissues (e.g., bone, muscle, nerves, cartilage, etc.) and exposed surfaces (e.g., skin, gums, teeth) of the anatomical portion. Non-radiographic scanning is performed on the exposed surfaces of the anatomical portion of the patient and the proximal end portion of a marker that is positioned in an opening formed in the anatomical portion of the patient. The marker may be a pin, a drill, an implant, a fiducial marker, or a screw, for example, and typically has a distal end portion disposed in the opening formed in the anatomical portion of the patient, with its proximal end portion projecting outwardly from the opening. A mid-operative 3-dimensional image is generated of the exposed surfaces of the anatomical portion and of exposed surfaces of the proximal end portion of the marker, as a result of the non-radiographic scanning of the anatomical portion of the patient and the proximal end portion of the marker. The mid-operative 3-dimensional image of the exposed surfaces of the anatomical portion and of the exposed surfaces of the proximal end portion of the marker is overlaid and aligned with the initial 3-dimensional image of the internal tissues and exposed surfaces of the anatomical portion of the patient, to produce an overlaid image. A 3-dimensional image representation of substantially the entire marker, including the proximal and distal end portions thereof, is obtained. The 3-dimensional image representation of substantially the entire marker is overlaid and aligned with the exposed surfaces of the proximal end portion of the marker that appear in the overlaid image. The 3-dimensional position of the distal end portion of the marker, relative to the internal tissues of the anatomical portion of the patient, is then visually confirmed via reference to the overlaid image.

According to one aspect, the step of scanning the anatomical portion of the patient to produce the initial 3-dimensional image thereof, includes performing at least one chosen from (i) an X-ray, (ii) a CT scan, (iii) a CBCT scan, and (iv) an MRI scan.

According to another aspect, the step of scanning the anatomical portion of the patient to produce the initial 3-dimensional image thereof, is performed prior to the step of creating the opening in the anatomical portion of the patient.

According to yet another aspect, the step of non-radiographic scanning the anatomical portion of the patient and the proximal end portion of the marker is an optical laser scanning step.

According to still another aspect, the marker is at least one chosen from (i) a pin, (ii) a drill, (iii) a surgical implant, and (iv) a screw.

According to a further aspect, the step of overlaying and aligning the mid-operative 3-dimensional image with the initial 3-dimensional image, includes aligning at least one fiducial marker that is visible in both the mid-operative 3-dimensional image and the initial 3-dimensional image. Optionally, the fiducial marker includes at least one chosen from (i) a tooth, (ii) an exposed portion of bone, and (iii) a portion of a surgical guide that is fitted to the anatomical portion of the patient.

According to a still further aspect, the opening in the anatomical portion of the patient is an osteotomy.

According to another aspect, the step of obtaining the 3-dimensional image representation of substantially the entire marker, includes at least one chosen from (i) selecting the 3-dimensional image representation of the marker from an electronic database, (ii) optically scanning the marker to create the 3-dimensional image representation thereof, and (iii) using ionizing radiation to scan the marker and create the 3-dimensional image representation thereof.

According to yet another aspect, the method further includes the step of attaching a surgical guide to the anatomical portion of the patient, the surgical guide configured to align a surgical tool that is used for the creating the opening in the anatomical portion of the patient.

According to another form of the present invention, a method is provided for evaluating the position of a marker relative to anatomical tissue in a medical operation. The method includes the steps of scanning an anatomical portion of a patient to produce a pre-operative 3-dimensional image thereof including a depiction of internal tissues; preparing a 3-dimensional physical model of the outer surfaces of at least a portion of the anatomical portion of the patient that corresponds to the scanned portion; positioning a marker in a desired location and orientation at the physical model; scanning the physical model to produce a 3-dimensional image of the model, in which at least a portion of the marker is captured in the 3-dimensional image of the model. The 3-dimensional image of the physical model is then overlaid and aligned with the 3-dimensional image of the physical model and the marker with the pre-operative 3-dimensional image of the patient anatomy, to verify the marker's position and orientation relative to the patient's internal tissues. After verification, a surgical guide that is configured to align a surgical tool with a location and orientation at the anatomical portion of the patient corresponding to the location and orientation of the marker at the physical model, is applied to the patient anatomy. A surgical operation is then performed to modify the anatomical portion of the patient using the surgical guide, the resultant modification to the anatomical portion of the patient substantially corresponding to the location and orientation of the marker at the physical model.

In one aspect, the step of preparing the 3-dimensional physical model includes at least one chosen from (i) performing an optical scan of the portion of the patient's anatomy without the use of ionizing radiation and creating the physical model from resulting optical scan data using rapid prototyping apparatus, (ii) performing an optical scan of a molded impression of the patient's anatomy and creating the physical model from resulting optical scan data using rapid prototyping apparatus, and (iii) using a molded impression of the patient's anatomy to create a cast thereof.

In another aspect, the step of scanning the physical model to produce a 3-dimensional image includes the use of non-ionizing radiation, so that only an exposed portion of the marker is captured in the 3-dimensional image of the physical model.

In still another aspect, the method further includes obtaining a 3-dimensional image of the marker, overlaying and aligning the 3-dimensional image of the marker with the exposed portion of the marker in the 3-dimensional image of the physical model.

In a further aspect, the marker includes a radiopaque material and the scanning the physical model to produce a 3-dimensional image thereof includes the use of ionizing radiation, wherein substantially the entirety of the marker is captured in the 3-dimensional image of the physical model. Optionally, the marker includes at least one chosen from a dental filling material, barium sulfate acrylic monomer, a pin, a drill, a surgical implant, a surgical guide, and a screw.

According to another form of the present invention, a method is provided for evaluating the accuracy of a surgical guide for use in creating an opening in body tissue. The method includes the steps of scanning a portion of a patient's anatomy to produce a pre-operative 3-dimensional image thereof including a depiction of internal tissues; preparing a physical model of the outer surfaces of a portion of the patient's anatomy corresponding to at least a portion of the scanned portion; creating an opening in the physical model; at least partially filling the opening in the physical model with a radiopaque material; scanning the physical model to produce a 3-dimensional image thereof, including the radiopaque material; and overlaying and aligning the 3-dimensional image of the physical model with the pre-operative 3-dimensional image of the patient anatomy to verify whether the opening created in the physical model is positioned as desired relative to the patient's internal tissues. Optionally, after the step including verification, a medical procedure may be performed in which the surgical guide is placed on the patient's anatomy, and an opening may be formed in the patient's anatomy using the surgical guide, the resultant opening substantially corresponding to the opening in the physical model.

In one aspect, the step of creating the opening in the physical model includes placing a surgical guide on the physical model and creating the opening in the physical model using the surgical guide.

In another aspect, the step of creating the opening in the physical model includes drilling a hole in the physical model, and the radiopaque material has a generally cylindrical shape as it fills the hole. Optionally, the radiopaque material includes at least one chosen from a dental filling material, barium sulfate acrylic monomer, a pin, a drill, a surgical implant, a surgical guide, and a screw.

According to another form of the present invention, a method is provided for pre-operatively evaluating the accuracy of a hole or an incision to be formed in body tissue. The method includes the steps of scanning a portion of a patient's anatomy to produce a pre-operative 3-dimensional image thereof including a depiction of internal tissues; preparing a first physical model of the outer surfaces of a portion of the patient's anatomy corresponding to at least a portion of the scanned portion; creating an opening in the physical model with reference to the pre-operative 3-dimensional image as a guide; at least partially filling the opening in the physical model with a radiopaque material; scanning the physical model to produce a 3-dimensional image thereof, including the radiopaque material; and overlaying and aligning the 3-dimensional image of the physical model with the pre-operative 3-dimensional image of the patient anatomy to verify whether the opening created in the physical model is positioned as desired relative to the patient's internal tissues.

According to another form of the present invention, a method is provided for evaluating the position of a marker relative to anatomical tissue in a medical operation. The method includes the steps of scanning a portion of a patient's anatomy to produce a pre-operative 3-dimensional image thereof, including a depiction of internal tissues; performing non-radiographic scanning of outer surfaces of the portion of the patient's anatomy including an exposed portion of a marker that is attached to the patient's anatomy, to create a 3-dimensional image thereof; obtaining a 3-dimensional image of the marker; overlaying and aligning the 3-dimensional image of the entirety of the marker with the exposed portion of the marker in the 3-dimensional image that includes the outer surfaces of the portion of the patient's anatomy to create a first composite image; overlaying and aligning the 3-dimensional image including the depiction of internal tissues with the combined 3-dimensional images of the outer surfaces of the patient's anatomy and the entirety of the marker to create a second composite image; and evaluating the position of the entirety of the marker relative to the internal tissues as shown in the second composite image.

Thus, the present invention provides techniques and systems that provide the benefits of mid-surgical 3-dimensional radiographic scans with a CT, CBCT, X-ray, MRI, or similar device, but without the mid-surgical use of ionizing radiation, and typically more quickly and at lower cost than would be the case when using equipment that produced ionizing radiation. The techniques may also be used to create accurate surgical guides, to confirm the accuracy of surgical guides prior to and/or during a surgical procedure, and to evaluate the result of the surgical procedure, all without the use of additional ionizing radiation. In addition, the techniques may be used without alteration to the preferred surgical methods and surgical equipment of a surgeon or other medical professional.

These and other objects, advantages, purposes and features of the present invention will become apparent upon review of the following specification in conjunction with the drawings.

### BRIEF DESCRIPTION OF THE DRAWINGS

FIG. 1 is a front (coronal) perspective view of a 3-dimensional computer tomography (CT) image of the right portion of a human lower jaw;

FIG. 2 is a top perspective view of a cast of a human lower jaw portion having test holes drilled therein corresponding to proposed osteotomies in a corresponding patient jaw for receiving dental implants;

FIG. 3 is a top perspective view of a cast of part of the right portion of a human lower jaw, having a test hole drilled therein, and with a cured radiopaque material filling the test hole;

FIG. 4A is a screen capture showing an image overlay in accordance with the present invention, in which a scanned 3-dimensional image of a cast of a human lower jaw (stippled) having a marker in the cast, is aligned and superimposed with a scanned 3-dimensional image of a corresponding human lower jaw portion shown at right, and with 2-dimensional top (axial), side (sagittal), and front (coronal) views of the overlay images shown at left;

FIG. 4B is another screen capture of an image overlay in which a scanned 3-dimensional image of a marker that is positioned in a physical model is superimposed with a scanned 3-dimensional image of a corresponding human lower jaw portion shown at right, and with 2-dimensional top (axial), side (sagittal), and front (coronal) views of the overlay images shown at left;

FIG. 5 is a top perspective view of a portion of the cast of FIG. 3, shown fitted with a surgical guide defining a guide hole aligned with the test hole that is formed in the cast;

FIG. 6 is a top perspective view of a right side portion of another cast, similar to that of FIG. 3, shown with a stud placed in a test hole that has been drilled in the cast;

FIG. 7A is a front perspective view of the cast and stud of FIG. 6, shown fitted with a surgical guide;

FIG. 7B is another front perspective view of the cast and surgical guide of FIG. 7A, shown with a drill guide tool fitted to the surgical guide at the guide hole;

FIG. 8 is a step-by-step diagrammatic representation of an image overlay technique in accordance with the present invention;

FIG. 9A is an initial misaligned 2-dimensional coronal cross-section image (hollow outline) from an optical scan of a 3-dimensional model, shown overlaid with a coronal 2-dimensional cross-section image (stippled) from a pre-surgical CBCT scan of a corresponding portion of a patient jaw;

FIG. 9B is an overlaid image of an aligned 2-dimensional coronal cross-section image (hollow outline) from an optical scan of a 3-dimensional model with a marker, shown overlaid with a coronal 2-dimensional cross-section image (stippled) from a pre-surgical CBCT scan of a corresponding portion of a patient jaw, and taken through section line 9B-9B in FIG. 8 at ‘H’;

FIG. 9C is an overlaid image of an aligned 2-dimensional coronal cross section image (hollow outline) from an optical scan of a 2-dimensional model with a marker, shown overlaid with a coronal 2-dimensional cross-section image (stippled) from a pre-surgical CBCT scan of a corresponding portion of a patient jaw, and taken through section line 9C-9C in FIG. 8 at ‘H’;

FIG. 10A is a perspective view of a mid-surgical 3-dimensional optical (laser) scan of the external surfaces of a patient jaw portion, in which a surgical region has a surgical drill protruding from an osteotomy and used as a marker;

FIG. 10B is a 3-dimensional sagittal reconstruction based on the optical scan of FIG. 10A, in which approximate tooth root portions and mandibular nerve are shown, and the entire drill is shown, including portions below the gum line, based on an image overlay of the exposed upper (proximal) end portion of the drill, so that the lower (distal) end portion of the drill is accurately extrapolated below the gum line;

FIG. 10C is a 2-dimensional coronal cross section of a CBCT scan of a patient jaw portion overlaid with an extrapolated 2-dimensional coronal cross section (outline) of a drill used as a marker and taken from a 3-dimensional optical scan similar to that of FIG. 10A;

FIG. 11A is a is a perspective view of a post-surgical 3-dimensional optical (laser) scan of the patient jaw portion corresponding to FIG. 10A, with the surgical region having a dental implant installed in (and protruding from) an osteotomy and used as a marker;

FIG. 11B is a 3-dimensional coronal partial cross-section reconstruction image of the dental implant and jaw portion of FIG. 11B, in which the lower portion of the dental implant has been extrapolated below the gum line and in which internal anatomical (jaw) tissues are shown, for evaluation purposes;

FIG. 11C is a 2-dimensional cross-section of a CBCT scan of a patient jaw portion overlaid with a 2-dimensional coronal cross section (line) of the gum tissue and exposed upper portion of the dental implant taken from the 3-dimensional optical scan of FIG. 11A, and a 2-dimensional coronal cross section (outline) of the full dental implant shown aligned with the exposed upper portion of the dental implant;

FIG. 12 is a hybrid diagrammatic view of a chair-side portable 3-dimensional scanning and image overlay system in communication with various data sources and a stereolithography machine, in accordance with the present invention;

FIG. 13 is a side perspective view of a dental implant supported between a radiolucent plate and a radiolucent upper support for use in scanning the dental implant for obtaining a 3-dimensional image thereof;

FIG. 14 is a side perspective view of a portion of a human spine having two surgically fused discs, and with pedicle screws driven into the adjacent fused discs, the screws being joined together by respective stabilizer rods;

FIG. 15 is a screen capture showing, at right, a 3-dimensional superimposed pedicle screw placement in a human spine (taken from behind), and with a 2-dimensional axial view of one of the superimposed pins shown at top-left, a 2-dimensional oblique side view of the superimposed pin shown at middle-left, and a 2-dimensional oblique top view of the superimposed pin shown at bottom-left;

FIG. 16 is another screen capture, similar to that of FIG. 15, showing 3-dimensional and 2-dimensional views of a superimposed pedicle screw placement in a human spine;

FIG. 17 is a screen capture showing a 3-dimensional rear perspective view of the exposed portions of pedicle screws placed in respective vertebrae the spine;

FIG. 18 is a screen capture showing a semi-transparent 3-dimensional rear perspective corresponding to FIG. 17, and showing the full pedicle screws relative to internal spine tissues; and

FIG. 19 is a top sectional view of a portion of a human torso in which a spine surgical guide has been attached to a vertebra for use in installing markers in the vertebra.

### DESCRIPTION OF THE PREFERRED EMBODIMENTS

The systems and image-overlay techniques of the present invention include devices and methods used to determine the precise anatomical position of surgical implants or prostheses, as well as osteotomies or other modifications to body tissues. 3-dimensional volumetric rendering software of pre-surgical patient DICOM (Digital Imaging and Communications in Medicine) image files are superimposed with either DICOM images of physical models or optical (such as laser) scans of patient anatomy or models, or negative DICOM images of an impression of the patient's anatomy, to accurately reveal precise anatomical or potential anatomical implant positions. The image-overlay techniques allow a clinician to fabricate, evaluate, and confirm accuracy of a surgical guide using computed tomography (“CT”) scanning, cone beam computed tomography (“CBCT”), laser scanning, or the like, prior to or during placement of a dental implant (or other surgical implant).

In accordance with current suggested radiologic guidelines of the ALARA principle, which is a system for limiting the radiation doses received by patients that is recommended by the International Commission on Radiological Protection (ICRP), the image-overlay systems and techniques described herein can significantly reduce patient exposure to radiation during and immediately after surgical procedures, without compromising diagnostic quality. It will be appreciated that the systems and techniques of this invention have other applications in dentistry and orthopedic reconstruction, and may be used, for example, in reconstructive surgery, lateral orbital decompression in ophthalmology, substantially any osteotomy or osteoplasty, veterinary surgical procedures, other medical procedures that involve altering (such as cutting or drilling) body tissues and/or placement of prostheses, implants, or the like, and for assisting in the orientation and alignment of radiographic devices, such as during oncological procedures.

The image-overlay techniques of the present invention limit a patient's exposure to radiation (particularly ionizing radiation) due to radiographic scanning, such as X-rays, CT-scans, CBCT-scans, and the like, while facilitating significant reduction in the time and the cost of fabricating a drilling or surgical guide, where desired, and enabling personnel at a medical office (such as a dental office) to prepare such guides in-house if they so choose. The systems, techniques and methods described herein can also improve the accuracy of the finished surgical guide and allow a dentist or surgeon or other medical professional to visually confirm that a hole (e.g., an osteotomy) drilled through the surgical guide will be in the proper location and in the correct alignment in the patient's jaw (upper or lower mandible) or other part of the body. Systems and methods are also provided for evaluating the position of an osteotomy or a marker in the patient's tissue, without the use of ionizing radiation.

As will be more fully appreciated with reference to the more detailed descriptions below, the image-overlay techniques can be implemented before surgery, during the process of placement of dental implants (or other surgical implants), and/or after surgical placement of dental implants (or other surgical implants). One application or benefit of the image-overlay techniques is the ability to create “CT-confirmed” in-office surgical guides, as will be described below. Other applications of the image-overlay techniques include mid-surgical and/or post-surgical evaluation procedures, which are described herein as a “pick-up technique” or laser-scanning corollary technique.

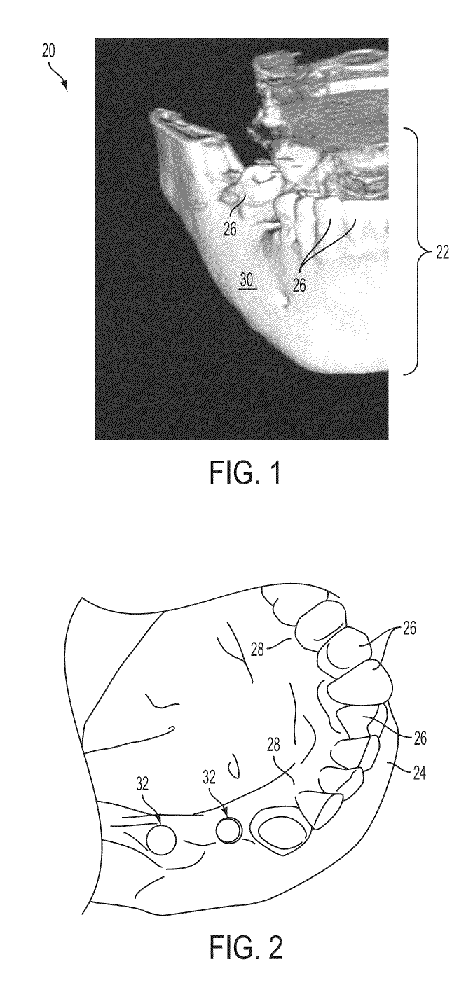

The techniques of the present invention may be used for evaluating the accuracy of a surgical guide, and/or for fabricating a surgical guide, for use in drilling an osteotomy in a dental patient's jaw that is receive a dental implant such as a false tooth. In referring to the appended drawings, it will be appreciated that the same reference numeral is generally used for a particular part of the anatomy or other device regardless of whether it is shown as a display image of the actual anatomical part, or as a representative image of a physical model or a scan of the model. Thus, for example, a tooth is designated with reference numeral 26 whether it appears as the CT-scanned image of a tooth (FIG. 1), or as the plaster cast of a tooth (FIG. 2).

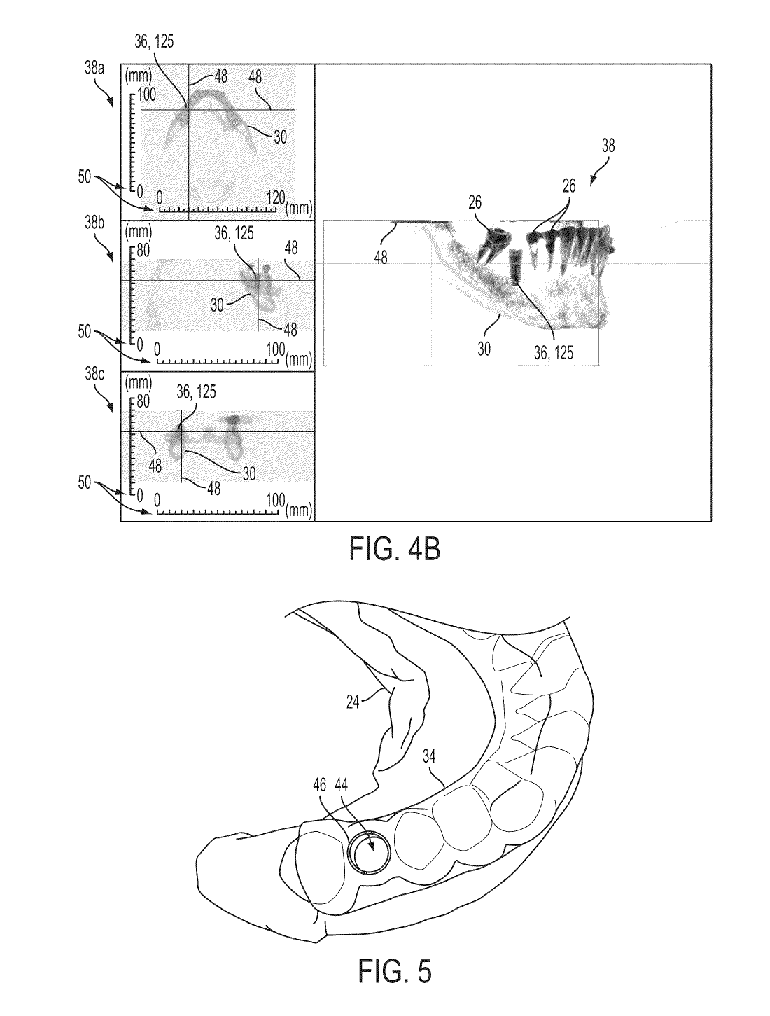

Regardless of the medical procedure that is underway or is being planned, the initial step of the image-overlay techniques of the present invention is typically the acquisition of an accurate diagnostic CBCT/CT-generated 3-dimensional DICOM (or other digital format) image file of the relevant anatomical portion of the patient, such as shown in FIGS. 1, 4A, and 4B, which is usually accomplished prior to any cutting or drilling of the patient's tissues, such as for placement of a dental (or other surgical) implant.

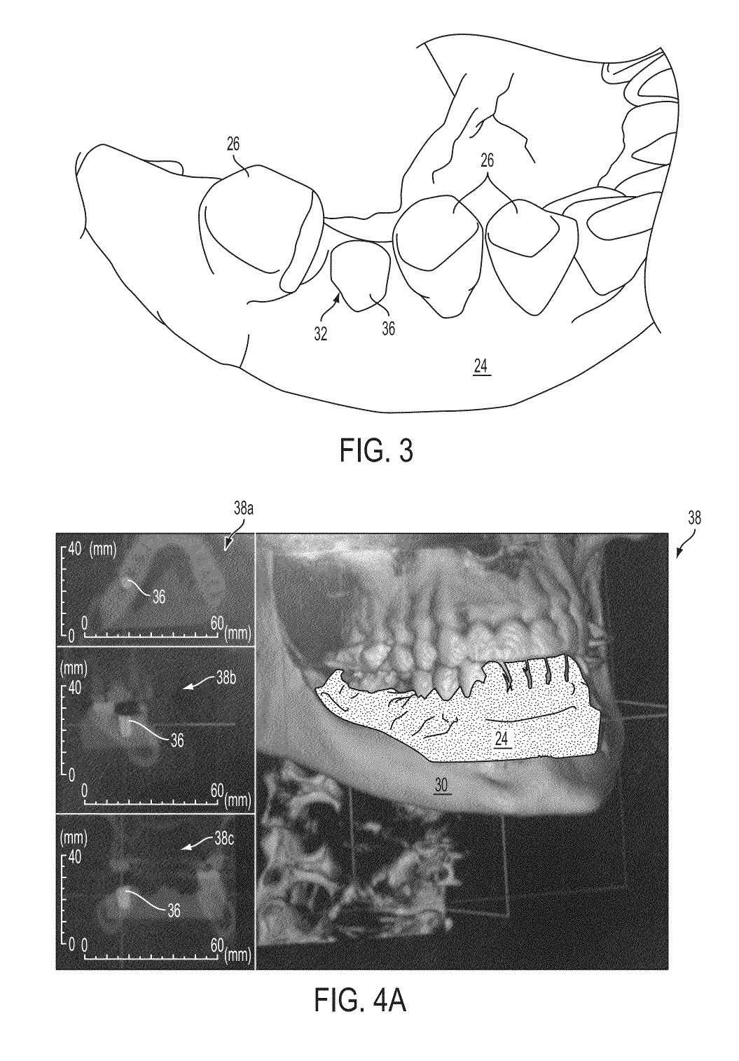

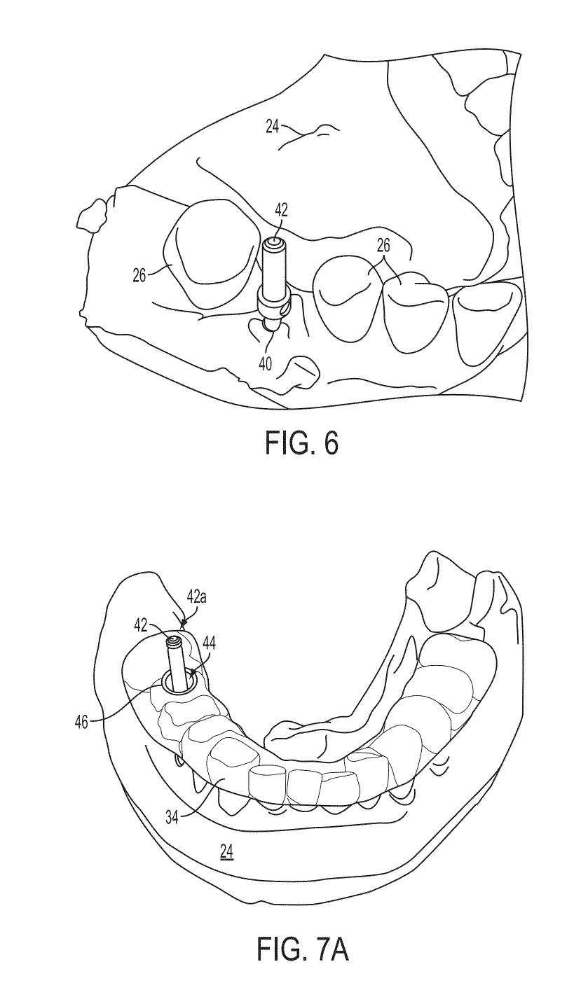

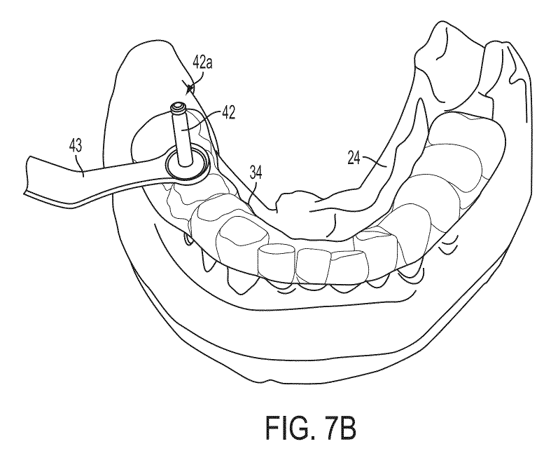

In one form, the technique is applicable to a dental implant procedure that generally follows these steps:
1) The dental professional captures a 3-dimensional CBCT-scan or CT-scan image 20 of the patient's jaw area 22 in need of a dental implant, such as shown in FIG. 1 and the non-stippled portions of FIG. 4A.
2) A physical model 24 or “diagnostic cast” or “wax up” (FIG. 2) is made of the patient's teeth 26 and gums 28 in the same area as CT-scan 20, to determine implant placement based on ideal restorative position. This model 24 can be fabricated with rapid-prototyping methods based on the CT-scan data, or based on a laser scan of the patient's teeth and gums, or can be fabricated as a cast (e.g. a plaster cast) taken from a mold of the patient's teeth 26 and gums 28, for example.
3) Based on the dental professional's knowledge of the location of the patient's jawbone 30, relative to the patient's teeth 26 and gums 28, based on a study of the tissues shown in the initial CT-scan of FIG. 1, the professional drills one or more holes 32 or “osteotomies” in the model or cast 24 (FIG. 2) in the desired 3-dimensional position (including the buccal-lingual position, mesial-distal position, and angle of insertion) for each dental implant. This drilling may be done free-hand, or may be performed using a drilling guide (or “surgical guide”) 34, such as shown in FIG. 5. The holes 32 are drilled in the model or cast 24 to the desired final diameter and depth, such as shown in FIG. 2. It will be appreciated that the final depth of each osteotomy 32 will vary due to tissue thickness, and is determined based on an image-overlay step that will be described below. At this point in the process, the clinician has a model with a hole (a simulated osteotomy) drilled in the physical model to the desired implant diameter and length for each proposed osteotomy.
4) The drilled hole in the model is then filled with a radiopaque marker 36 (FIG. 3), which may be a dental filling material (composite), barium sulfate acrylic monomer, a metallic stud or pin or drill bit, or the like.
5) Another 3-dimensional CBCT-scan or CT-scan is made of the model/cast 24 (not of the patient), in which the radiopaque marker 36 in the drilled hole 32 is clearly visible in the image generated by this scan of the model/cast 24. Except for the presence of a radiopaque material-filled hole 32 and the lack of internal anatomical detail, the image produced by this second radiographic scan (of the model/cast) will be substantially identical to the image 20 produced by the first scan (of the patient's actual jaw area), because the surfaces of the model/cast are substantially identical to the surfaces of the patient's actual jaw area.
6) A software program is used to digitally overlay the second scan with the first to form a composite image 38 (FIGS. 4A and 4B). The first CT-scan image (of the patient's anatomy, having smooth grey surfaces in FIG. 4A) and second CT-scan image (of the model 24, having stippled surfaces at right in FIG. 4A) can be precisely aligned with one another on-screen, because the patient's anatomy will be substantially identical in each of the overlaid images, which are set at a 1:1 size scale. However, the radiopaque material-filled hole 32 from the second scan will appear superimposed in the patient's jaw, such as best shown along the left side portion of FIG. 4A (i.e., front sectional view 38a, side sectional view 38b, and top sectional view 38c). A similar second scan image may be produced by implanting a surgical implant 126 in the model 24 and using it as a radiopaque marker 36, such as shown in FIG. 4B. In the overlaying or superimposition step, the patient DICOM images (first scan) and the prepared and working cast images (second scan) may be superimposed in precise alignment by aligning anatomical landmarks or fiducial markers, such as for procedures involving soft-tissue-borne surgical guides. Optionally, superimposed cross-hairs 48 and length measurement scales 50 may be shown on the sectional views 38a-c (FIG. 4B) to aid the clinician in determining placement and orientation of the radiopaque marker 36 in the model 24, relative to the patient's internal tissues shown in the initial radiographic scan image. Suitable software programs for performing image overlays may include Invivo software, available from Anatomage Inc., of San Jose, Calif.
7) Once the 3-dimensional composite image is set with proper alignment, the dental professional or other technician can manipulate the composite images of FIGS. 4A and 4B on-screen, to view them from substantially any desired angle or perspective, such as coronal (front) plane view 38a, sagittal (side) plane view 38b, and axial (top) plane view 38c, such as shown along the left side portions of the images as shown in FIGS. 4A and 4B. These views reveal the planned anatomical path of the implant, but typically do not account for the thickness of the exposed or outer soft tissue architecture, such as gums, which would affect osteotomy depth but not the drilling orientation or path. Because the hole (simulated osteotomy) in the model 24 is filled with a radiopaque marker 36 (FIG. 3), the contrast and brightness of the 3-dimensional volumetric images can be adjusted to distinguish the radiopaque marker 36 (or “virtual implant”) from the material of the model 24 and the underlying anatomy visible in the pre-surgical patient CBCT or CT scan. The image of the radiopaque marker 36 contrasts the anatomical structures of the patient (i.e.: teeth, bone, major nerve bundles, sinuses, or other necessary landmarks of concern) in the composite images of FIGS. 4A and 4B, and allows diagnostic assessment of the proposed implant placement and analysis of a fabricated surgical guide. Thus, the dental professional can use the composite image 38 to determine whether a given surgical guide 34 will provide proper alignment for a proposed drill hole made using the guide 34, or to determine whether the drilled hole 32 in the model 24 is in the desired location, as will be described in more detail below.
8) If the drilled hole 32 in the model 24 is found to be satisfactory based on the composite overlay image 38, and was made without the benefit of a surgical guide, a new surgical guide 34 can be made from the model/cast 24, such as shown in FIGS. 5-7B. To produce the surgical guide 34, at least a portion of the radiopaque marker is removed from model 24, such as by drilling a pilot hole 40 into the center of the radiopaque marker 36 (FIG. 6), inserting a guide element 42 (such as an insert handle, a sleeve, an indexing pin, or a stud) in the pilot hole 40. As shown in FIG. 7A, an upper end portion 42a of the guide element 42 projects outwardly from the pilot hole 40 and can be used to precisely position a surgical guide hole 44 that may be lined with a metal ring 46 in surgical guide 34, which is typically made of a resinous plastic material that is heated and conformed over the representative teeth 26 of the model/cast 24 by applying downward pressure, as is known in the art.
9) The surgical guide 34 may then be placed over the patient's teeth (similar to its placement on model 24, as shown in FIG. 5), and the surgical guide hole 46 will be at the precise location and alignment so that an osteotomy drilled through the guide hole 44 in the surgical guide 34 and into the patient's jaw bone, will be in substantially the identical location in which the radiopaque marker 36 appeared in the composite image 38. The drilling of the osteotomy through the surgical guide 34 may typically involve the use of a surgical guide tool 43 that is seated in the guide hole 46 for aligning a drill 52, such as shown in FIG. 7B.

As briefly noted above, a similar technique may be used to evaluate the accuracy of an existing surgical guide 34, whether that guide has been made using the technique(s) described herein, or by other methods. The surgical guide 34 is fitted to the model/cast 24 of the patient's teeth, and a hole 32 (simulated osteotomy) is drilled into the cast or model 24 using the surgical guide 34. The hole 32 is filled with radiopaque marker 36, scanned, and the resulting image is overlaid with the original patient CT scan in substantially the same manner as described above. It will be appreciated that substantially any surgical guide can be evaluated for accuracy prior to surgery using the techniques describe herein. For example, if a clinician fabricates an in-office surgical guide or orders a commercially-prepared surgical guide, and desires to test the accuracy of that guide, the clinician may repeat the pre-surgical phase of the technique on a duplicate model with a new radiopaque marker to assess the precision and accuracy of the surgical guide. When the clinician is satisfied with the virtual implant (radiopaque marker) position provided by the image-overlay technique, the surgical guide may be sterilized and then used during surgery on the patient.

As described above, one pre-surgical benefit of the image-overlay techniques of the present invention is the ability to fabricate an accurate in-office “CT-confirmed” surgical guide, while another benefit is to permit or facilitate confirmation of the accuracy of substantially any surgical guide regardless of its fabrication method. Although there are differences in preparation, the outcomes and benefits of producing pre-surgery “CT-confirmed” surgical guides are substantially similar. By employing the image-overlay technique and reviewing the results, if a commercially-prepared surgical guide is deemed acceptable to the clinician, the image-overlay technique provides a means to “CT-confirm” the precision of substantially any fabricated surgical guide.

Different manufacturers of surgical drilling systems typically utilize a different “V-factor” for their surgical drills or “burs” (i.e., defining the portion of the bur that is included in dimensional calculations provided by the bur manufacturer), which should be taken into consideration during implant placement. When osteotomy depth is a concern, such as due to encroachment upon a “safety zone” of an adjacent vital anatomical internal structure (such as neurovascular bundle, maxillary sinus, or cortical plate perforation), the use of optional techniques (such as the “pick-up technique” or its laser-scanning corollary, described below), may be more appropriate. Such techniques facilitate the avoidance of errors during the surgical phase of treatment, which may be particularly challenging to address and correct. Therefore, the use of techniques and systems that facilitate a dental professional's ability to quickly and accurately assess osteotomy preparation and final implant position can be very important.

### Pick-Up Technique

Optionally, a dental professional may take steps to further ensure proper location of a hole (osteotomy) that is drilled into the patient's jaw 30, by using a “pick-up” technique that involves placing a stud in an initial pilot hole drilled in the jaw (similar to pilot hole 40 drilled in model 24, such as shown in FIG. 6), and taking an impression of the region during the surgical procedure of placing the insert or prosthesis. When the impression material cures in the patient's mouth and is removed, the stud stays in the impression material and thus is removed (“picked up”) from the pilot hole formed in the patient's jawbone. The impression material is then used to make a cast or model (such as a plaster cast), and the cast will include the stud (or a drilled hole filled with a radiopaque marker representing the stud). The cast is then scanned to create a 3-dimensional image that is overlaid with the original 3-dimensional patient scan (showing internal tissues) to verify whether the pilot hole in the patient's jaw is placed in the desired location and orientation. It will be appreciated that the making and scanning of a cast or other physical model with the stud is optional, since the impression itself could be scanned and viewed as a negative, which would result in an image that is the equivalent of a scanned image of a “positive” mold or model made from the “negative” impression of the patient's anatomy.

Regardless of whether the clinician has a “CT-confirmed” surgical guide, a traditional CT-guided surgical guide, or an in-office fabricated surgical guide, the pick-up technique (or its laser-scanning corollary technique, described below) is useful for avoiding multiple scans of the patient using ionizing radiation. Using the image-overlay techniques described herein, a modified indexing pin or any other marker or object that can be captured or partially encapsulated in a pick-up impression (or that is capable of being optically distinguished by a 3-dimensional laser scanner), during or after surgery, can serve as a useful diagnostic adjunct. A captured indexing pin or analog for each osteotomy (or an implant or implant abutment, if the impression is taken post-placement) is partially encapsulated in the impression material, in the location that accurately represents the location of the pin, implant, marker or analog in the patient's anatomical tissue. At this point, based on time and availability of access to a CBCT or CT scan, the clinician can evaluate a negative image of the impression superimposed with the pre-surgical scan, or can pour a model of the impression and perform a scan followed by the same type of superimposition steps described above. However, it will be appreciated that laser scanning of the area of the patient undergoing surgery can be used in place of a pick-up impression, with image extrapolation techniques used to indicate the depth and orientation at which the indexing pin (or other object positioned in the osteotomy to serve as a marker) extends into the patient's tissue (such as jaw bone).

Accordingly, the pick-up technique (and the laser-scanning corollary technique described below) allows for precise evaluation of multiple implant placements at the same time during surgery, without relying on 2-dimensional representations of implant placements from periapical or panoramic films. When the clinician uses the pick-up technique during the early stages of osteotomy preparation, errors in angulations, depths or location can be identified early and adjustments can be made, typically without compromising treatment outcomes. It will be appreciated that use of a pick-up impression or laser scan of the patient's anatomy during surgery can result in the patient having no additional exposure to radiographic scans such as X-ray, CT scan, CBCT scan, or other ionizing radiation during or immediately after surgery, while still permitting a mid-surgical assessment of the placement of multiple implants. Thus, the pick-up technique or its laser-scan corollary (described below) provides a beneficial clinical procedure that follows the recommendations of the ALARA principle by offering a significant advancement compared to existing surgical and radiologic protocols.

### Laser-Scanning Corollary to Pick-Up Technique

The laser-scanning technique permits evaluation of the position of a marker (e.g., an implant or screw) position in 3 dimensions, during or after surgery, with no ionizing radiation exposure to the patient or surgical staff, and without the use of an impression of the patient anatomy. Like the above-described pick-up technique, the laser-scanning technique reduces or eliminates the need for the use of mid-surgical or immediate post-surgical X-ray images to evaluate single or multiple implant or surgical fixation screws. Although primarily described herein as a “laser scanning” technique, it will be appreciated that substantially any optical or non-radiographic scanning technique may be used, as long as it is capable of generating 3-dimensional digital images of the outer or exposed surfaces of an anatomical portion of the patient, such as teeth, gums, skin, or internal tissues (e.g., bone, muscle, tendons, cartilage, blood vessels) that are exposed during a surgical operation.

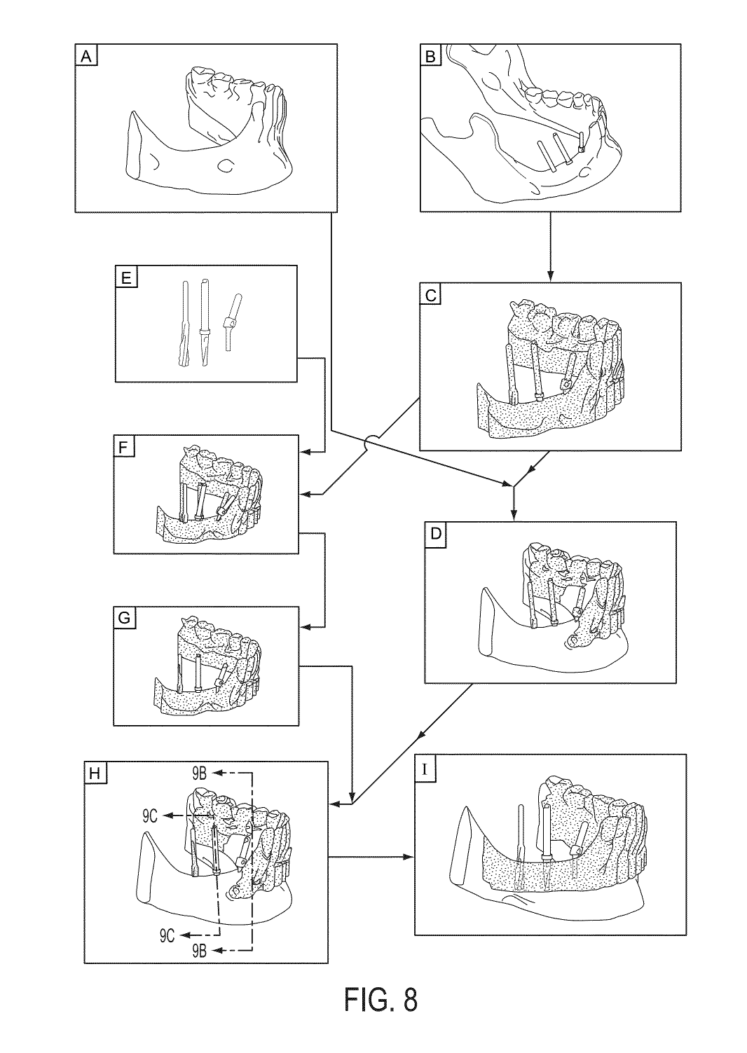

The basic steps of the laser-scanning technique are illustrated diagrammatically in FIG. 8 and described immediately below, while a more detailed description of the technique will follow. The laser-scanning technique involves a double superimposition process in which three or more 3-dimensional digital images are obtained and combined in stages. A. Initially, a pre-operative DICOM (or other digital format) 3-dimensional image is obtained (FIG. 8, at ‘A’), typically via X-ray scan, CT scan, CBCT scan, MRI scan, or other (typically radiographic) imaging method. A surgical procedure is then performed by medical personnel to install one or more markers in the patient anatomy, or an analog procedure is performed on an accurate model (such as a plaster cast) of the anatomical region of the patient, such as shown in FIG. 8 at ‘B’, in which three surgical markers have been inserted into respective holes formed or established in a model of the patient jaw portion. An optical (e.g., laser) or non-radiographic scanned image (FIG. 8, at ‘C’), such as a laser Virtual Surface Anatomy Scan Image (such as in a stereolithography or “STL” 3-dimensional image format), is made of the outer or exposed anatomical surfaces of the patient (or of the model corresponding to the patient anatomy), including exposed portions of any markers present. The markers may be substantially any object (e.g., a pin, a drill, an implant, a surgical guide or appliance having a fiducial marker, or a screw) that is capable of being optically or non-radiographically scanned by an electronic image scanning device, and that is visually differentiable from surrounding tissues or other surfaces. The resulting 3-dimensional laser-scanned image is represented by stippled surfaces in FIG. 8 at ‘C’, ‘D’, and ‘I’ through ‘T’.

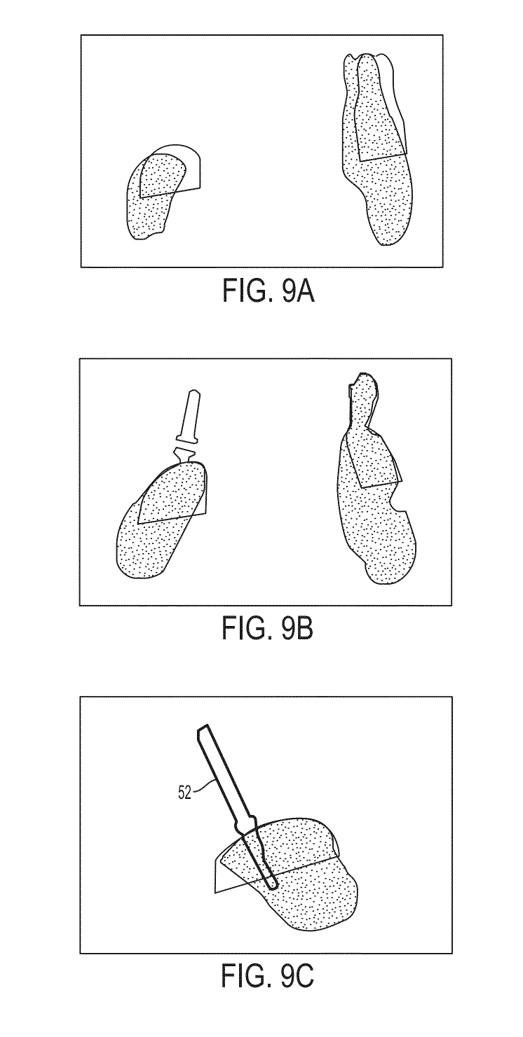

The pre-operative image of the patient anatomy (shown in FIG. 8 at ‘A’ and represented by non-stippled surfaces in FIG. 8 at ‘D’, ‘H’, and ‘I’) and the laser-scanned image (represented by stippled surfaces in FIG. 8) are converted to compatible digital image formats (e.g., STL or DICOM) if necessary, and are combined or superimposed or overlaid and aligned with one another to form a first composite image (FIG. 8, at ‘D’). The alignment step resulting in the image of FIG. 8 at ‘D’ may be facilitated with reference to one or more cross sectional views, such as the coronal cross-section view of FIG. 9A in which the laser-scanned surface image (hollow outline in FIG. 9A) is overlaid with the pre-surgical CBCT scan (stippled in FIG. 9A) at a corresponding portion of the patient jaw. Once the laser-scanned image of FIG. 8 at ‘C’ is obtained, digital images of the corresponding one or more markers (FIG. 8, at ‘E’) may be superimposed therewith to create another composite image (FIG. 8 at ‘F’). In the composite image of FIG. 8 at ‘F’, the non-stippled images of the entire markers are initially misaligned with the exposed proximal or upper end portions of the markers (stippled) of the laser-scanned image. However, it will be appreciated that the lower portions of the entire markers (non-stippled) may be obscured by the laser-scanned image at this stage, as shown in FIG. 8 at ‘F’. The technician can then individually manipulate the image of each entire marker on-screen to achieve proper alignment of its upper exposed end portion with the upper exposed end portion of its match in the (stippled) laser-scanned image, such as shown in FIG. 8 at ‘G’, in which stippled and non-stippled image portions are visibly intermingled.

At this stage in the image overlay and evaluation process, two aligned composite images have now been prepared, the first composite image being that of FIG. 8 at ‘D’ in which the laser-scanned outer surfaces of the patient anatomy are overlaid and aligned with the pre-operative image, and the second composite image being that of FIG. 8 at ‘G’ in which the entire marker images are overlaid and aligned with the exposed portions of the markers appearing in the laser-scanned image. These two composite images may now be combined or overlaid or superimposed with one another to form a third composite image (FIG. 8 at ‘H’), in which the properly-aligned markers are shown with the laser-scanned image of the exposed anatomical surfaces, which are aligned with the radiographically-scanned image of the same region. For clarity of illustration, the radiographically-scanned image and the laser-scanned image have been shown as opaque where they appear in the drawings of FIG. 8 at ‘A’ and at ‘C’ through ‘H’. However, it will be appreciated that these images may be readily made at least partially translucent, such as shown in FIG. 8 at ‘I’, which would include a depiction of internal tissues (not shown in FIG. 8) in the case of the radiographically-scanned image, so that the technician or medical professional can visually verify or study the location of the lower or distal (embedded) portion of each marker relative to those internal tissues, for evaluative purposes. The resulting double-superimposed composite image of FIG. 8 at ‘I’ reveals accurate bony anatomy, soft tissue anatomy, and marker (drill, implant, stud, etc.) positions relative to those tissues, without the use of multiple radiographic scans of the patient, and without taking an impression of the patient's jaw portion or other anatomical region. Optionally, 2-dimensional cross sectional views may be generated along different planes in the 3-dimensional images of FIG. 8 at ‘H’ and ‘I’, such as shown in FIGS. 9B and 9C.

It is envisioned that the order of at least some of the steps may be altered from the manner in which they are described above, and that the steps themselves may be altered to some degree, without departing from the spirit and scope of the present invention. For example, the 3-dimensional images of the entire markers, as shown in FIG. 8 at ‘E’, could be overlaid or superimposed directly into the first composite image of FIG. 8 at ‘D’, to arrive at substantially the identical third composite image of FIG. 8 at ‘H’ (and, thus, of the corresponding translucent image of FIG. 8 at ‘I’), without need for a separate step of generating the composite image of FIG. 8 at ‘G’, in which the entire marker images are overlaid and aligned with the exposed portions of the markers appearing in the laser-scanned image.

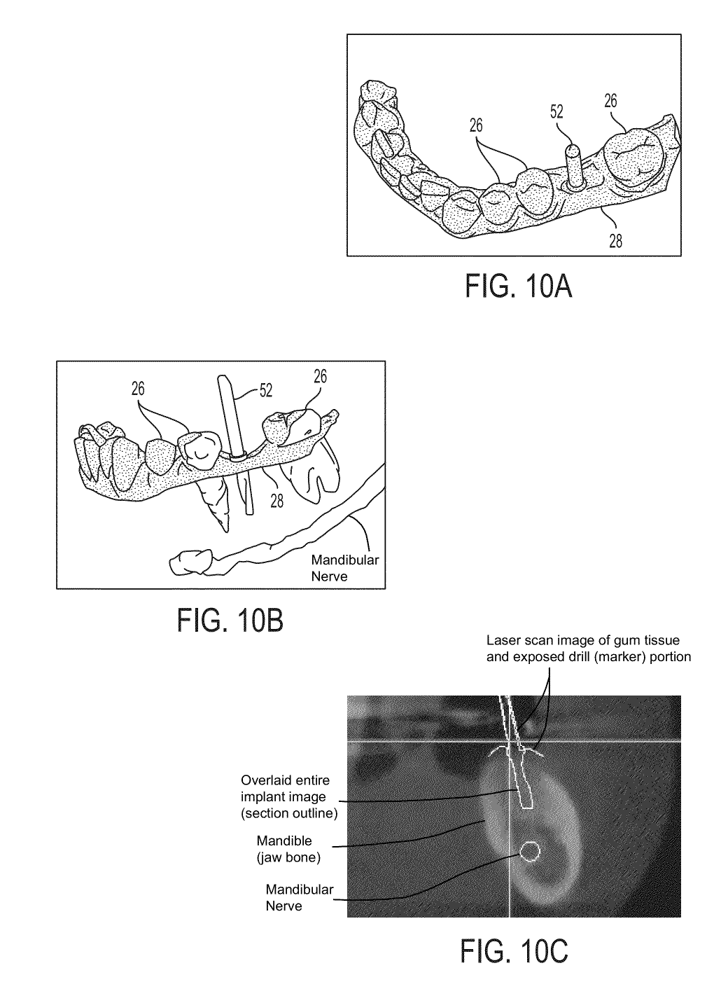

The laser scan described above results in a 3-dimensional digital image showing only the outer or exposed surfaces of the scanned anatomical portion of the patient, such as the patient's jaw area, including gums and teeth, with the stud or implant (marker) positioned in the pilot hole or in a final osteotomy, such as shown in FIG. 8 at ‘C’ and in FIG. 10A. The marker's dimensions are known from manufacturer data or from scanning the stud itself prior to its implantation and, preferably, a 3-dimensional image is available (or obtainable through scanning), which depicts the outer surfaces of substantially the entire marker. If 3-dimensional marker images are not available from the manufacturer of the marker or another source, such images may be obtained using an optical scanner (such as the same laser scanner that is used to generate images of the exposed surfaces of the patient anatomy) to create an STL or DICOM format (or other format) image of the marker(s) to be used during the medical procedures. Optionally, it is desirable to create a library of 3-dimensional images of an assortment of different markers that are readily available for access by a computer used in the image-overlay process. This may be particularly helpful, for example, when the type of marker being used is changed during the surgical process.

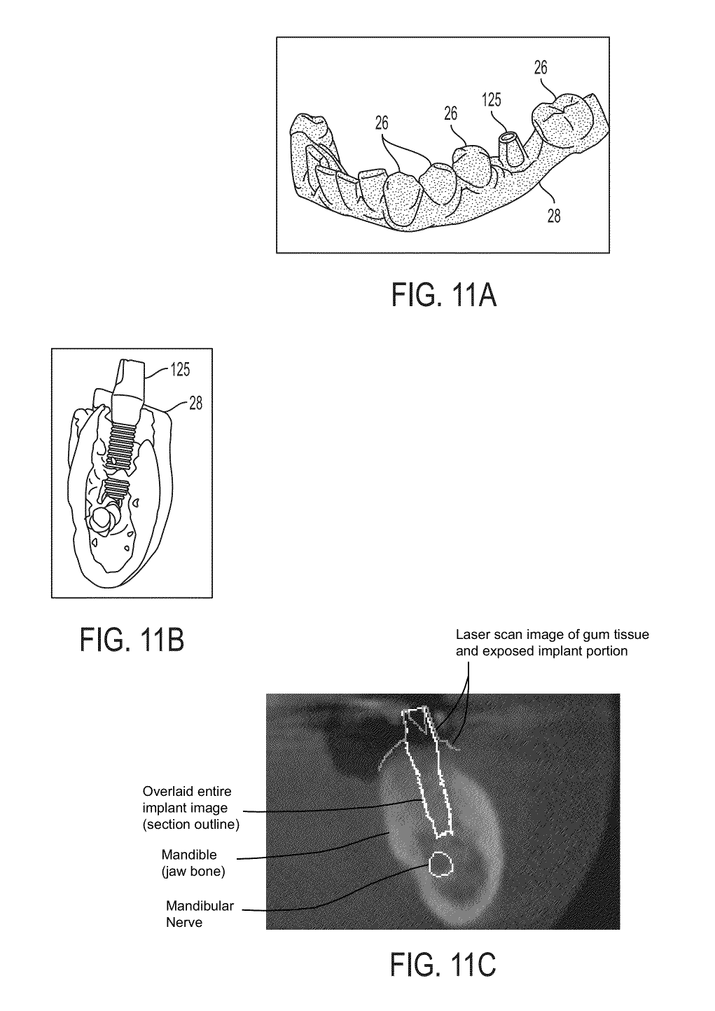

As described above, the dimensions or 3-dimensional images of the marker(s) (FIG. 8 at ‘E’) allow a technician to create an overlaid or composite image that accurately represents the depth and orientation of each marker's lower or distal portion (which is inserted into the osteotomy in the patient's jaw) relative to the patient's internal anatomical tissues, such as shown in FIGS. 10B-10C and 11B-11C. This is typically accomplished by overlaying and aligning the upper portion of the 3-dimensional image of substantially the entire marker with the upper portion of the marker that is exposed above the gum line in the laser-scanned image. The image of the lower (distal) portion of the marker thus projects or is extrapolated below the exposed tissue surfaces that were scanned by the laser scanner, such as shown in FIGS. 8 at ‘I’ and 10B. The laser scanned image with the overlaid image of the entire stud can then be overlaid with the original CT scan of the patient's jaw area, showing internal tissues such as bone and nerves (e.g., FIGS. 10C, 11B, and 11C), so that the dental professional can visually verify whether the osteotomy in the patient's jaw has been drilled at an appropriate orientation and depth, with the resulting composite image being viewable from substantially any desired angle for viewing from different vantage points.

Surgical drills or other surgical instruments (indexing pins, etc.) with known dimensions and shapes can also be used as markers and captured in the laser-scanned image of the patient's exposed anatomical surfaces, and extrapolated as described above, as long as there is a digital image of the drill or instrument being used as a marker. The markers used in the image overlay techniques are preferably rigid or substantially rigid so that the markers cannot be flexed or otherwise distorted during normal use, in order to facilitate the image overlay techniques described herein.

The laser-scanning technique will now be described in more detail, including optional steps. In a pre-surgical or initial phase of medical procedure in which overlay imaging is to be used, the following steps may be followed:
(1) A 3-dimensional image of the relevant patient anatomy (e.g., a CT, CBCT, MRI, other equivalent diagnostic image) is obtained, such as shown in FIG. 8 at ‘A’, but with internal tissues made visible as needed;
(2) the ideal or desired placements of implants, screws, or other medical devices are planned based on a pre-surgical plan;
(3) a physical model is made of the patient anatomy, such as by pouring a cast of an impression, or by stereolithography or other rapid-prototyping technique;
(4) the marker(s) are installed in the model (FIG. 8 at ‘B’), according to the pre-surgical plan;
(5) the model with marker(s) installed is scanned with a laser scanner (or equivalent scanner using non-ionizing radiation) to create a Virtual Surface Anatomy Scan (3-dimensional image) that includes the model representation of the skin or gums, the surgical marker(s), and teeth, such as shown in FIG. 8 at ‘C’, prior to any surgery on the patient;
(6) a 3-dimensional image of the entire marker (which may be a pin, drill, an implant and/or related components, a screw, etc.) is created or obtained from another source (FIG. 8 at ‘E’);
(7) the 3-dimensional image of the entire marker (or markers) is overlaid and aligned with the Virtual Surface Anatomy Scan (FIG. 8 at ‘F’ and ‘G’);
(8) a composite image is created by superimposing the original 3-dimensional patient image with the Virtual Surface Anatomy Scan (FIG. 8 at ‘D’);
(9) a final composite image of the original 3-dimensional patient image, the Virtual Surface Anatomy Scan, and the 3-dimensional image of the entire marker(s) is created (FIG. 8 at ‘H’ and ‘I’); and
(10) the medical professional evaluates the final composite image to determine where the markers, which at this point are positioned only in the physical model of the patient's anatomy, would lie relative to the internal tissues of the patient's anatomy, if they were attached to the patient in precisely the same manner in which they are attached to the model.
(6) Optionally, a surgical guide may be fabricated at the model, such as in a manner described above, or obtained from another source, if a surgical guide is desired for use during the surgical phase of the procedure.

In the surgical or mid-operative phase of the laser-scanning technique, the following steps are generally followed:
(1) Optionally, a surgical guide with fiducial markers (i.e., distinguishable “landmarks” that can be used as an alignment aid) is placed on the patient anatomy to aid in superimposition (e.g., for edentulous cases);
(2) surgery is performed as usual according to the individual surgeon's preferences;
(3) any time a mid-surgery evaluation is desired, a laser scan (or equivalent scan using non-ionizing radiation) is taken of the exposed anatomical surfaces and any markers in the surgery area, such as shown in FIGS. 10A and 11A, to create a Virtual Surface Anatomy Scan that results in an image showing patient skin or gums, the surgical guide(s) or marker(s) present, and teeth, such as shown in FIGS. 10A and 11A, and similar to what is shown in FIG. 8 at ‘C’ (which is actually a laser-scanned image of the surfaces of a model corresponding to the patient anatomy);
(4) the 3-dimensional image(s) of the entire marker(s) (which may be any sufficiently rigid drill, implant, screw, or pin of substantially any shape) are superimposed and aligned with the protruding portions of the marker(s) visible in the mid-surgery laser scan, and evaluated for desired placement of the marker(s) in the composite image that includes the interior tissues shown in the pre-surgical image, such as shown in FIGS. 10C and 11C;
(5) optionally, the alignment of the Virtual Surface Anatomy Scan with the pre-surgical image may be accomplished with reference to overlaid cross-sections of the images, such as shown in FIGS. 9A-9C;
(6) optionally, the surgeon may make changes at the surgical site based on information obtained from the composite image(s) in the preceding step(s);
(7) optionally, additional optical scans are made of the surgical site until the surgical procedure is complete; and
(8) optionally, post-operative optical scans and overlays are used to confirm that the final implanted device(s) is/are in the desired locations and orientations, such as shown in FIGS. 10B, 10C, 11B, and 11C.

### Additional Considerations

It will be appreciated that both the pick-up technique and its laser-scanning corollary can be used to place multiple studs, implants, or markers in a patient's jaw during the same overall procedure, and may be accomplished while the patient undergoes a single dental or other medical procedure, rather than during multiple procedures spaced out over several days or weeks. As noted above, and as will be more fully appreciated with reference to additional descriptions provided below, these procedures may be used not just in dental procedures, but in substantially any medical procedure in which an implant or prosthesis is to be attached to human tissue, particularly bone. However, it is envisioned that the technique could also be used in connection with softer tissues like cartilage or any other tissue that can be distinguished on CT, MRI or equivalent 3-dimensional imaging.

It will also be appreciated that the image-overlay techniques of the present invention depend at least somewhat on (i) a medical professional's (or imaging technician's) ability to successfully and accurately superimpose 3-dimensional volumetric rendered data for implant placement, (ii) discrepancies between the depth of the virtual dental implant placement (e.g., in a physical model) and final implant position in the patient anatomy, and (iii) the time required during mid-surgery to evaluate a superimposition during execution of the pick-up technique, for example.

The ability to successfully superimpose 3-dimensional volumetric rendered data accurately may warrant additional attention in totally edentulous cases (patients without any teeth), for example, as it may be desirable for a radiopaque stent (or a guide with radiopaque fiducial markers) to be worn by the patient, during the original radiographic scan. However, as long as the original scan utilizes a radiopaque stent or the like, the accuracy of the image overlay techniques should remain substantially the same as for cases in which there are natural fiducial markers or landmarks (e.g., teeth) present. A duplicate denture with barium sulfate acrylic monomer (or the like) with fiducial markers, which can be superimposed after the osteotomies are drilled, may also be helpful for superimposition. The replication of tooth-borne surgical guides can be facilitated by taking precautions to reduce the amount of scatter radiation, and the teeth may be separated so that superimposition of the dentition (teeth arrangement) is possible.

The potential for discrepancies between the depths of the virtual implant (e.g., in a physical model, or a pilot hole or osteotomy in a patient's anatomy) and final implant position in the patient anatomy can be readily addressed. While the thickness of a surgical guide may be negligible with respect to predicted implant placement, it is desirable for a clinician to be aware of necessary anatomical considerations and have knowledge of the surgical system used to accurately approximate osteotomy depth during surgery. The pick-up technique (or its laser scanning corollary) provides the clinician with the ability to accurately assess the state of surgery for multiple implants, if necessary. While conventional periapical films could assist the clinician with depth approximation, the pick-up technique or laser scanning corollary described herein may be used for determining the depth of a given osteotomy without the use of ionizing radiation or other radiographic imaging.

During mid-surgery, the time required to take a pick-up impression (or laser scan of the patient's mouth portion), create a DICOM image, superimpose the pre-surgical and pick-up impression image (or 3-dimensional laser image), and then perform evaluation, may be a concern. However, it is envisioned that the time expended by a clinician to address failed implant placement post-surgery would typically be considerably greater than using the pick-up technique (or laser scanning corollary) to verify placement during surgery. This can be addressed, for example, by immediately using the pick-up impression to create a negative DICOM image, or by using a laser-scanning method to create an image model of the surgery area. In the case of creating a negative DICOM image, imaging software should be capable of superimposing a negative DICOM image and an original patient scan. The faster processes may utilize, for example, a macro or micro CT unit or 3-dimensional high definition (HD) laser scanner that is portable, cost effective, and provides substantially immediate or automatic superimposition without manual superimposition of DICOM images, such as will be described below in more detail.

As previously mentioned, the image-overlay techniques of the present invention are equally applicable to other areas of dentistry and medical surgery. For example, the pick-up technique and its laser-scanning corollary technique can permit clinicians to accurately assess placements of temporary anchorage devices in orthodontics and post placement for endodontically-treated teeth in restorative dentistry. It is possible that obturation or endodontic materials could be captured in a pick-up impression and superimposed to also confirm accuracy. By further example, airway volumes can be visualized using these methods, with superimposition used to determine the effect of dental sleep appliances. The techniques may also be used by orthopedic surgeons, such as for knee, hip, or other long bone reconstructions, if adequate pre-surgical images are taken prior to surgery. It will be appreciated that modifications may be needed to accurately utilize the pick-up impression technique in non-dental environments, particularly those in which natural fiducial markers (such as teeth) are not present, but in general the same overall methods would be used outside of a dental environment.

It is envisioned that the techniques described herein may also benefit medical professionals undergoing training and/or evaluation, by allowing clinicians to be objectively assessed during training and early surgical procedures. Accurate feedback and evaluation of clinical skill would be beneficial to students learning to place dental implants, which could result in reduced clinical failures, improved surgical outcomes, and reduced radiation exposure to patients.

Therefore, image-overlay techniques of the present invention can improve the quality of patient care with evidenced-based 3-dimensional evaluations of substantially any surgical guide, and can also provide a method or technique for obtaining immediate or rapid feedback/confirmation of the surgical placement of dental implants or the like. While the use of surgical guides improves the probability that desirable outcomes will be achieved, these outcomes are at least somewhat dependent on individual clinician skill levels and judgment, surgical conditions, available equipment, and different techniques, so that mere use of a surgical guide does not guarantee predictability or success. The image-overlay techniques of the present invention can help to reduce these variables and thus reduce clinical failures, improve surgical outcomes, and reduce radiation exposure to patients and medical professionals. Images similar to those obtainable using CT, CBCT, and MRI equipment may be obtained with greater speed and lower cost, and without the use of additional ionizing radiation, and the surgical techniques and surgical equipment used during surgery need not be altered in order to make use of the medical image evaluative techniques of the present invention.

### System for Use in Image-Overlay Techniques

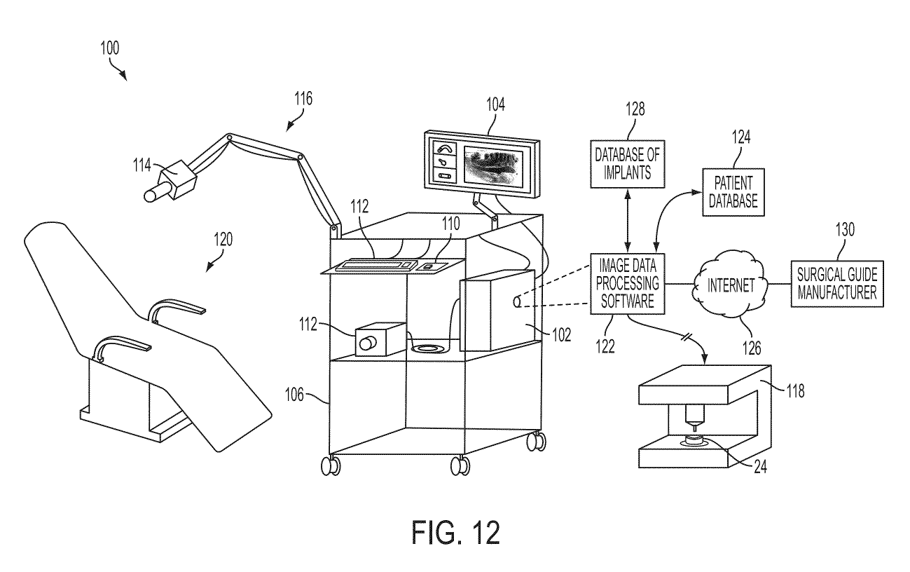

Referring now to FIG. 12, an image-overlay medical evaluation system 100 includes a computer 102 with associated display 104. In the illustrated embodiment, these components are supported or mounted on a portable cart 106, although it will be appreciated that the individual components of system 100 need not be supported together. A standard keyboard 108 and mouse 110 or other controllers (e.g., a touch-screen) are in communication with computer 102 and mounted at a location on portable cart 106 (such as on a tray, as shown) to provide convenient access by a medical technician.

Computer 102 is in communication with several other peripheral devices including a macro CBCT scanner 112, a laser scanner 114 (shown coupled to cart 106 via an articulated arm 116), and a rapid prototyping machine 118 for producing diagnostic models 24. When the image-overlay medical evaluation system 100 includes a portable cart 106 as shown, the computer 102, display 104, keyboard 108, and mouse 110, macro CBCT scanner 112 and laser scanner 114 may be transported together around a medical office, such as for use adjacent a dental or medical chair 120, while computer 102 may be in communication with its peripheral devices via wired or wireless connections.

Computer 102 operates a software package 122 for processing images and data, and for communicating images and data via wired or wireless connections (FIG. 12). For example, software 122 can be used by the medical clinician to operate macro CBCT scanner 112 and laser scanner 114, and to manipulate, convert, and overlay images collected by the scanners 112, 114 on display 104. Since laser scanner 114 and macro CBCT scanner 112 may produce images in different formats, it is desirable that software 122 be capable of converting one or more image formats into another one or more different formats, so that the images collected by different devices can be displayed together in an overlying fashion, such as in the manner described above. Suitable laser scanners capable of scanning anatomical surfaces may include, for example, those currently manufactured by Northern Digital Inc. (“VicraScan”) and Basis Software Inc. (“Surphaser”), as well as the Cadent iTero intraoral scanner, available from Align Technology, Inc. of Carlstadt, N.J., and laser scanners available from NextEngine, Inc. of Santa Monica, Calif. Suitable CBCT scanners may include, for example, those manufactured by Carl Zeiss Industrielle Messtechnik GmbH (“METROTOM”), or the i-CAT scanner available from Imaging Sciences of California. Other devices that may be used for measuring dimensions include coordinate measuring machines (CMM's), such as those available from Hexagon Metrology, Inc. (“ROMER Absolute Arm”).

Thus, software 122 is configured to access, display, convert, and manipulate digital images in various formats including, for example, DICOM images, CAD images, STL images, or the like, such as may be generated by a digital laser scanner (e.g. scanner 114) or CBCT scanner (e.g., macro CBCT scanner 112). Software 122 permits a clinician to review digital images, visualize virtual models and create images overlays on display 114, and which may be saved in a patient database 124. In addition, software 122 may be operable to create and transmit laboratory prescriptions, such as digital models of anatomical features, to an on-site or off-site laboratory for use in fabricating a prosthetic (e.g., partial dentures, implant abutments, orthodontic appliances, and the like), surgical guides, or the like. Software capable of at least superimposing or overlaying images include Mimics software, available from Materialise NV of Leuven, Belgium, with image analysis facilitated by 3-Matic software, also available from Materialise NV.

Software 122 is in communication with patient database 124 (FIG. 12), which includes a collection of images (e.g., 3-dimensional or 2-dimensional images of patient anatomy, of models of patient anatomy, photographs, etc.) as well as substantially any other information relevant to a given medical patient (medical records, identifying information, etc.). Patient database 124 includes digital electronic archives of patient dental records including, for example, individual arch models, orthodontic appliances, dental prostheses, articulated models, and the like. Database 124 may reduce or eliminate the need to store physical diagnostic models, while facilitating access to digital images or models to create a stereo-lithographic model (or other rapid prototyping model) on an as-needed basis. Patient database 124 may be stored on a computer hard drive at computer 102, or may be stored on a remote drive, server, or the like, which may be located in or near the medical office, or at an offsite location, and accessed by computer 102 operating software 122 via wired or wireless communications. For example, patient database 124 may be administered by a third party service provider, and accessed and maintained via the Internet 126.

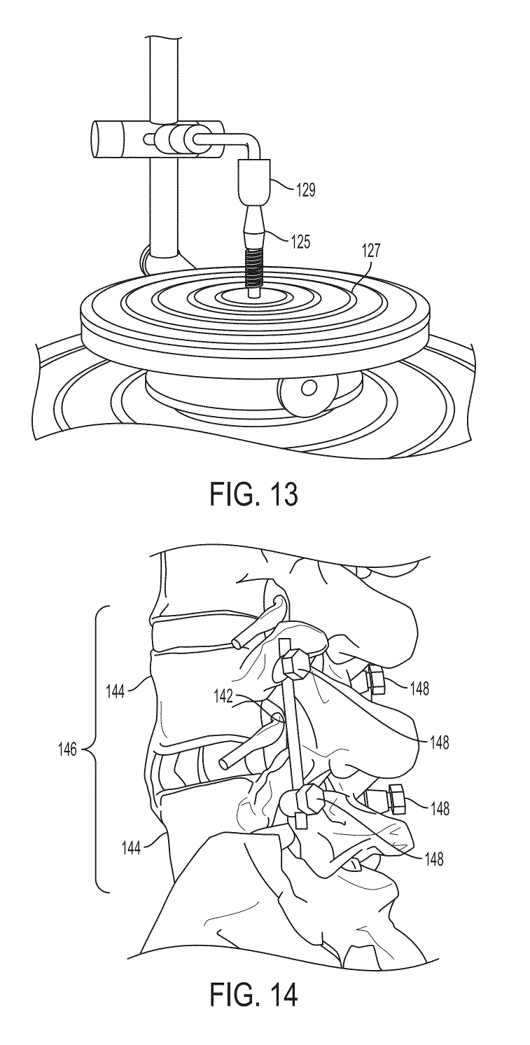

Optionally, software 122 may be in further communication with a marker or implant database 128 of medical implants and/or tools and/or prostheses (at least some of which can be used as markers), which includes dimensional information for a range of medical implants, tools, or the like, so that a properly-scaled rendition or image of a given implant or tool may be superimposed on the image(s) at display 104. Implant database 128 may include a radiographically or non-radiographically scanned image of a single marker, or a collection or “library” of markers. Optionally, software 122 may receive implant or tool information for implant database 128 via manufacturer-supplied data, or from 3-dimensional scanned data received from macro CBCT scanner 112 and/or laser scanner 114, which are in communication with computer 102. For example, and as shown in FIG. 13, an implant 125 is supportable between a radiolucent tray 127 and a radiolucent upper support 129 so that an accurate 3-dimensional representation of implant 125 may be obtained and stored in implant database 128 by scanning it with CBCT scanner 112 or another scanning device. Suitable scanners for implants 125 may include HD 3-dimensional scanners available from NextEngine of Santa Monica, Calif., as well as Maestro 3D, available from AGE Solutions of Pisa, Italy.

Optionally, software 122 is capable of creating custom graphical laboratory prescriptions for use in prosthodontic, orthodontic, implant, and other restorative dental procedures. Digital models can be virtually articulated (i.e., adjusted or oriented on-screen to ensure accurate alignment of an upper and lower jaw model, for example, to replicate the accurate bite and closed-jaw position of a patient) by a dental laboratory technician so that dental models can be super-imposed or overlaid on a 3-dimensional CBCT scan image, for example.

Optionally, images and/or data processed or managed by software 122 may be forwarded to a surgical guide manufacturer 130 or other third party recipient via the Internet 126 or other electronic data network. Software 122 may also be in direct communication with rapid prototyping machine 118 to produce physical models 24, with the rapid prototyping machine 118 located in the same medical office as the rest of medical evaluation system 100 (FIG. 12), or with the machine 118 located at an off-site location and accessed or controlled via Internet 126 or other electronic communications.

Image-overlay medical evaluation system 100 may be utilized in implant dentistry, such as for pre-surgical planning for dental surgical guides and verification of the accuracy of such guides such as using the techniques described above, or for orthodontics, restorative dentistry, CAD/CAM dentistry, the archiving of dental models, and the creation and storage of digital dental laboratory prescriptions. For example, in implant dentistry, system 100 may be used to verify the 3-dimensional position of surgical drills, dental implants, or substantially any other markers, such as described above with reference to the image-overlay techniques. Use of system 100 in orthodontics may include verifying the 3-dimensional position of surgical drills, temporary anchorage devices (TAD's), or other markers. The system 100 may be used in restorative dentistry, such as for verifying post or pin position in three dimensions. The ability to archive digital dental models permits or facilitates the creation of a “virtual library” of patient models, such as may be stored at patient database 124. The creation of digital dental laboratory prescriptions may include digital electronic copies of patient anatomy models and images for use in fabricating surgical guides, dental appliances, and the like.

When no surgical guide or computer-assisted surgery technique is used (e.g., when a dental professional chooses to free-hand drill osteotomies and placement of dental implants), laser scans of the surgical area may be taken before and after placement of the dental implants and the scanned images superimposed with one another. The superimposition of images may be more difficult if “flap surgery” is used (i.e., pulling back a portion of the patient's gums or other soft tissues to access jaw bone) or if no obvious anatomical landmarks are present, or if bone grafting, extractions, or alveoloplasty (the removal of bone tissue to smooth or re-contour the jaw bone) is completed at the time of implant placement. However, if teeth are present in the surgical area and are not changed during surgery, then the super-imposition of images may be significantly easier to accomplish. Optionally, for patients presenting edentulous (toothless) cases, a surgical guide with fiducial markers may be held in place in the patient's mouth via fixation screws.

When a surgical guide is used, laser scans of the surgical area may be taken before and after placement of dental implants and super-imposed with one another. The use of a surgical guide facilitates a super-imposition or overlayment of images, even if flap surgery is used, and even if no obvious anatomical landmarks are present, or if bone grafting, extractions, or alveoloplasty are completed at the time of implant placement. As with free-hand surgery, when teeth are present in the surgical area and are not changed during surgery, the superimposition of images will be made easier. In the case of a dentureless surgical area or procedures involving extractions, large incisions, or modifications to the alveolar ridge may make it more difficult to super-impose “before” and “after” images of the surgical area.

Thus, the image-overlay medical evaluation system 100 can significantly limit or reduce the amount of ionizing radiation exposure to a patient and dentist, surgical team, or other medical personnel. In addition, the ability to quickly determine the 3-dimensional position of markers such as implant drills, and to determine the final implant position, without use of ionizing radiation, can reduce post-operative complications and surgical failure, while reducing the likelihood that additional surgeries will later be required to address complications from the initial surgical procedure.

Optionally, the image-overlay medical evaluation system 100 may be utilized during other medical procedures, such as non-dental surgeries or the like (as in the spinal surgery example described below), without departing from the spirit or the scope of the present invention. For example, a mobile or fixed-position image-overlay medical evaluation system may be positioned in an operating room in a hospital or other medical facility, and used to facilitate the manual or automated orientation and super-imposition of 3-dimensional images of a surgical area, such as for use by a surgeon, radiologist, or a trained imaging technician involved in the surgery, to permit visualization of 3-dimensional or cross-sectional images during surgical procedures, and substantially without the use of ionizing radiation.

Depending on its operating environment, image-overlay medical evaluation system 100 may be designed to facilitate sterilization or disinfecting processes without damage to the individual components of the system. In addition to the ability of the image-overlay medical evaluation system 100 to facilitate the verification and/or identification of the 3-dimensional position and orientation of markers such as surgical drills, medical implants (implant devices, screws, other markers), or the like, the system may reduce equipment costs for hospitals and other medical providers, and improve the ability of smaller hospitals or medical facilities to obtain or utilize data from higher cost equipment through digital communications with the operators of such equipment, while reducing ionizing radiation exposure to patients and following radiation safety guidelines.

### Spinal Surgery Example of Image-Overlay Technique

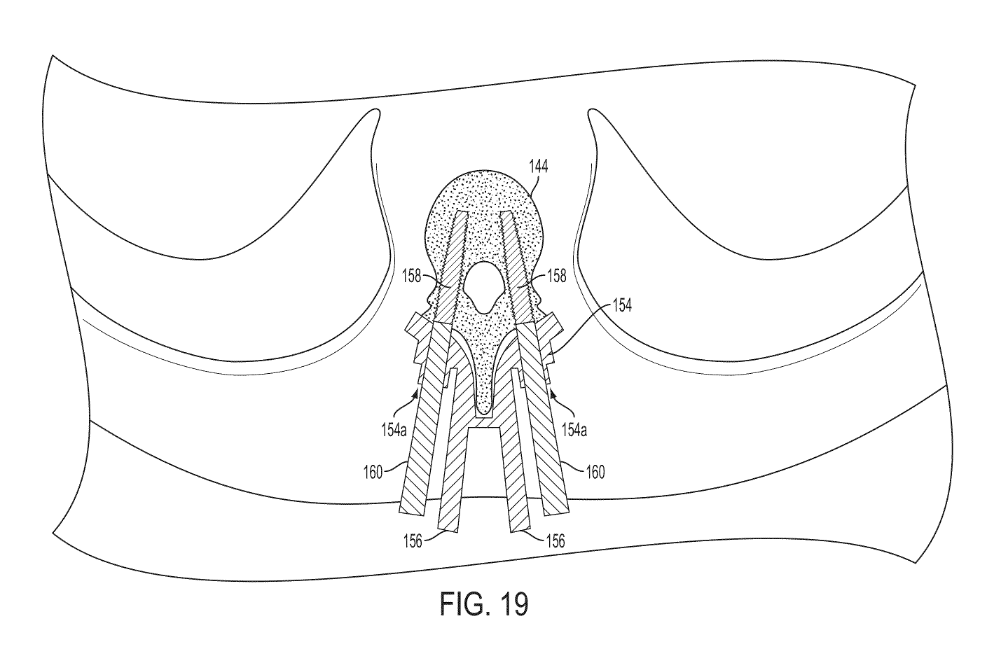

It will be appreciated that the image overlay techniques of the present invention, which are described above primarily in the context of dental implant surgery, may be practiced in connection with other types of surgeries or medical procedures to help ensure proper placement of implants or prosthetic devices, tools, or the like. For example, and with reference to FIGS. 14-18, the process of installing spinal disc-supporting pedicle screws 140 and stabilizer rods 142 to stabilize an adjoining pair of fused vertebrae 144 in a section of spine 146, can be facilitated by using the above-described methods to evaluate the placement of the pedicle screws 140 in the vertebrae 144 prior to actually drilling holes in the patient's vertebrae. Optionally, this procedure may further be facilitated by preparing a surgical guide to assist in drilling holes to receive pedicle screws 140, such as will be described with reference to FIG. 19. It will be appreciated that a surgical guide for placement of pedicle screws would be different in shape but substantially the same in principle as the dental surgical guides described above.

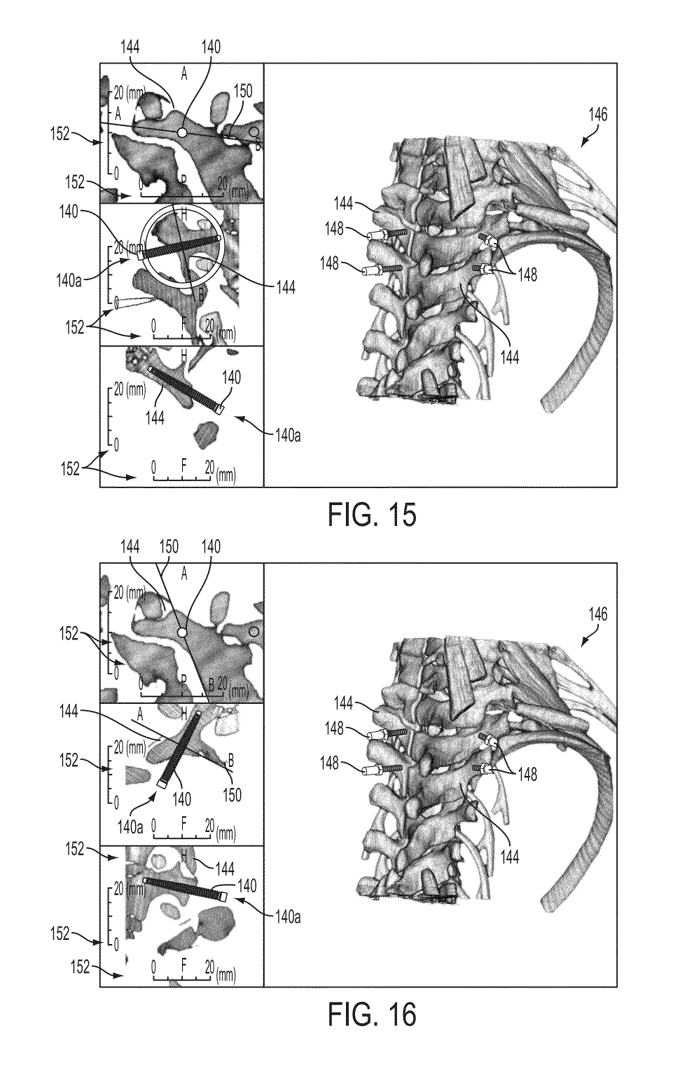

Imaging software, such as software 122 described above, may be used to generate 2-dimensional and 3-dimensional representations of the patient's spine section 146 with image representations of pedicle screws 140 superimposed with the image of spine section 146. For example, in FIGS. 14, 17, 18 and the right-hand portions of FIGS. 15 and 16, 3-dimensional outer surface image representations of spine section 146 include the exposed distal end portions 140a of a pair of pedicle screws 140 in each of two adjacent vertebrae 144, with respective head attachments 148 (for receiving stabilizer rods 142) shown attached at the distal end portions 140a in FIG. 14 and the right-hand portions of FIGS. 15 and 16.

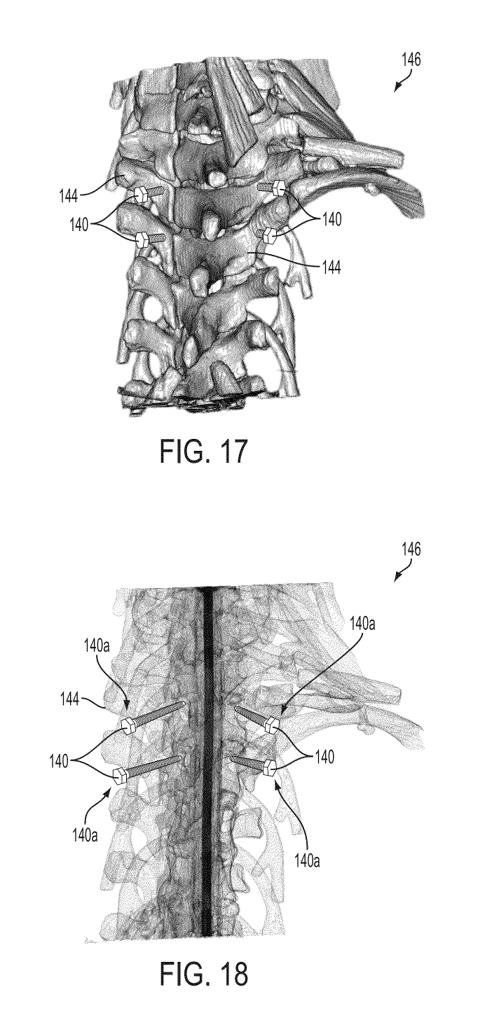

A clinician may manipulate the images of the individual pedicle screws 140 relative to the images of the patient's corresponding vertebrae 144, such as in the semi-transparent overlay image of FIG. 18, to ensure that the image representations of pedicle screws 140 are positioned in solid bone of the vertebrae and not too close to other tissues that could be damaged by the screws or by drilling. As best shown along the left-hand sides of FIGS. 15 and 16, the image software may generate axial and two different lateral sectional views (the lateral views being orthogonal to one another), corresponding to the right-hand 3-dimensional images, of a given pedicle screw placement in the image representation of the corresponding vertebra 144. Optionally, cross-hairs or alignment lines 150 and measurement scales 152 may be displayed in at least the 2-dimensional sectional images so that the clinician can readily measure the dimensions, alignment, and spacing for a proposed pedicle screw placement, such as to facilitate the methods described herein.

Once the clinician is satisfied that all of the image representations of pedicle screws 140 are in desirable locations in the image representations of the vertebrae 144, the clinician can prepare a physical model of the vertebrae and screws for use in preparing a surgical guide 154 having one or more guide holes 154a corresponding to each desired pedicle screw 140 or markers 158 (e.g., drills), such as shown in FIG. 19. Optionally, the surgical guide 154 can be tested on the physical model of the vertebrae by using it to drill pedicle screw holes into the model vertebrae using the surgical guide, and then scanning the model (such as with a CBCT scanner or laser scanner or the like) to determine if the surgical guide is sufficiently accurate before it is used to drill osteotomies in the patient's vertebrae for receiving the actual pedicle screws.

Optionally, the patient spine and pedicle screws or other markers (e.g., drills 158 with extensions 160, as in FIG. 19) can be laser-scanned during surgery to evaluate the placement of the osteotomies formed in the vertebra 144, as will be described in more detail below. This technique can reduce the size of the surgical area and the time required for the actual surgical procedure on the patient, while reducing the use of ionizing radiation on the patient and increasing the likelihood of a successful outcome for the patient.

One preferred method of spine surgery using a surgical guide, optical scanning, and image-overlay techniques generally utilizes the following steps, in which only one single image utilizing ionizing radiation technology (X-ray, CT, CBCT, etc.) is captured of the patient anatomy (surgical site) including internal tissues (e.g, bone, cartilage, muscle, tendons, nerves, etc.), prior to conducting surgery. Once a pre-surgical image depicting internal tissues has been obtained, a computer-aided design (“CAD”) plan may be used to place images of desired implants in desired locations (e.g., avoiding nerves and other soft tissues) in the pre-surgical image. A surgical guide 154 may be fabricated based on the desired implant locations determined in the preceding step, such as by using one of the guide-fabrication methods described above. In the illustrated embodiment of FIG. 19, surgical guide 154 includes a pair of elongated fiducial markers 156 that facilitate subsequent image-overlay steps.

Markers 158 (e.g., drills, implants, etc.) are selected corresponding to the implant images that were used in the CAD planning step, and appropriate implant extensions 160 are selected corresponding to the markers 158. The patient surgery is begun in a substantially conventional manner with an incision and any additional steps necessary to expose the vertebra 144 (or multiple vertebrae) that are planned to receive the marker(s) 158. Generally, the markers 158 are sufficiently long so that they will be at least partially exposed (e.g., projecting from the vertebra bone) when fully inserted therein. Surgical guide 154 is seated against the vertebra 144, such as shown in FIG. 19. Optionally, an initial pilot hole (osteotomy) is then drilled into the vertebra 144 through each guide hole 154a. If necessary, the osteotomies are enlarged until they are sized to receive the respective markers 158, although it will be appreciated that if a pilot hole is drilled, the drill that is used to form the pilot hole may itself be used as a marker during subsequent non-radiographic (e.g., optical or laser) scanning.

The marker 158, once implanted in vertebra 144, is optically scanned without use of ionizing radiation (e.g., via a laser scanner) to create a 3-dimensional image of the surgical area including the surgical guide 154 with fiducial markers 156, and marker extensions 160. The scanned image of the surgical guide and marker extensions has images of the markers 158 added to it, which is possible since the dimensions of markers 158 are known, as is the positioning of each marker 158 relative to its corresponding extension 160. The combined image of the surgical guide 154, implant extensions 158, and implants 158 are then overlaid with the pre-surgical image to show where the markers 158 are located within vertebra 144, such as shown in FIG. 19. Once the osteotomy locations have been verified as correct or acceptable, and the osteotomies are at their final desired sizes, the final implants may be positioned in the respective osteotomies, and the surgical procedure completed according to normal practices.

Optionally, the accuracy of the surgical guide 154 may be confirmed prior to drilling any osteotomies in vertebra 144, in a manner that is substantially similar to that described above with respect to a dental implant procedure. Once the surgical guide 154 has been initially positioned along the vertebra 144 mid-surgery, the implant extensions 160 may be positioned in guide holes 154a of surgical guide 154. The surgical area (including surgical guide 154 with its fiducial markers 156, and the portions of implant extensions 160 that project outwardly from guide holes 154a) is then optically scanned to create a 3-dimensional image. Images of the markers 158 (e.g., the final implants) are added and aligned precisely according to their known positions relative to implant extensions 160 to form a composite image. The composite image is then overlaid with the pre-surgical image, so that the marker images are projected into the tissues in the surgical site according to the positions in which the markers would be expected to be located when subsequently using surgical guide 154 to form osteotomies through guide holes 154a.

This technique has use in various procedures in orthopedics, such as substantially any surgical involving the fusion of vertebrae (spinal fusion, scoliosis treatment, trauma, etc.). From the pre-operative CT or MRI or CBCT, replicas of the patient's vertebrae involved with spinal fusion may be created from stereolithographic models (or other physical models made using known prototyping methods). Accordingly, the software allows the surgeon to plan the appropriate selection and placement of pedicle screws. A custom surgical guide for each vertebra may be fabricated to fit over the spinous process of the respective vertebral body. The spine will allow for custom placement on each vertebra without concern for the degrees of freedom of each individual vertebra, regardless of the surgical position of the patient. The surgical guide may or may not be fixated through any portion of the vertebra (if fixating is desired, this may be done through the spinous process) to limit displacement. In addition, it is envisioned that a plurality of surgical guides may be configured to interlock with one another, such as to assist in spinal fusion, if desired. Pedicle screws could then be drilled and placed through the pre-planned holes through the surgical guide or guides. The surgical guide can be made visible or used with extensions to help orient and facilitate superimposition using the image overlay techniques of the present invention. Pedicle screw extensions or marker extensions may also be helpful in applying the image overlay techniques.

Thus, the present invention provides systems and methods or techniques for medical image analysis, which can reduce the time and expense required for various medical procedures, while increasing the accuracy and/or allowing visual confirmation of the procedures. Visual confirmation may be performed prior to the actual medical procedure being performed on the patient, or may be performed mid-procedure and/or post-procedure. Other than an initial scan that is typically performed using X-ray, CT-scan, CBCT-scan, or MRI, visual confirmation of osteotomy or marker placement may be performed substantially without the use of radiographic scans, and without compromising the quality of the evaluation or outcome of the procedure.

Changes and modifications in the specifically described embodiments can be carried out without departing from the principles of the present invention, which is intended to be limited only by the scope of the appended claims, as interpreted according to the principles of patent law including the doctrine of equivalents.

The embodiments of the invention in which an exclusive property or privilege is claimed are defined as follows:

1. A medical imaging display system for collecting and displaying medical images, said system comprising:
a software program configured to enable the manipulation and overlaying a plurality of digital images;
a computer configured to execute said software program;
a display in communication with said computer;
a radiographic image scanning device in communication with said computer;
a non-radiographic image scanning device in communication with said computer;
a medical patient information database for storing patient medical images generated by said radiographic image scanning device and said non-radiographic image scanning device, wherein said medical patient information database is accessible by said computer;
a medical implant database for storing dimensional information for at least one medical implant, wherein said medical implant database is accessible by said computer; and
wherein said software program is executable by said computer to overlay and align a plurality of 3-dimensional images at said display, the 3-dimensional images including (i) a first 3-dimensional image of an anatomical portion of a patient that has been collected by said radiographic image scanning device, (ii) a second 3-dimensional image of the anatomical portion of the patient that has been collected by said non-radiographic image scanning device, and (iii) a 3-dimensional image of the at least one medical implant.

2. The system of claim 1, further comprising a rapid prototyping machine in communication with said computer and operable to create 3-dimensional physical models based on image data received from said computer.

3. The system of claim 1, wherein said radiographic image scanning device comprises at least one chosen from (i) an X-ray, (ii) a CT scan, (iii) a CBCT scan, and (iv) an MRI scan, and wherein said non-radiographic image scanning device comprises an optical laser scanner.

4. The system of claim 1, wherein said software program is operable to obtain the 3-dimensional image of the at least one medical implant directly from one chosen from (i) said radiographic image scanning device, (ii) said non-radiographic image scanning device, and (iii) said medical implant database.

5. The system of claim 1, wherein said non-radiographic image scanning device is configured to generate the second 3-dimensional image from either of (i) the anatomical portion of the patient or (ii) a physical model of the anatomical portion of the patient.

6. The system of claim 1, wherein said software program is configured to individually scale the sizes of the plurality of 3-dimensional images at said display, whereby each of the plurality of 3-dimensional images is scalable to correspond to the scale of others of the plurality of 3-dimensional images.

7. A method of evaluating the position of a marker relative to anatomical tissue, said method comprising:
scanning an anatomical portion of a patient to produce an initial 3-dimensional image thereof, including a depiction of internal tissues at the anatomical portion;
performing non-radiographic scanning of outer surfaces of the anatomical portion of the patient, including an exposed portion of a marker at the anatomical portion of the patient, to create a second 3-dimensional image thereof;
obtaining a 3-dimensional electronic image representation of the marker;
overlaying and aligning the 3-dimensional image representation of the marker with the exposed portion of the marker in the second 3-dimensional image, to thereby create a first composite image on a display;
overlaying and aligning the first composite image with the initial 3-dimensional image including the depiction of the internal tissues, to create a second composite image on the display; and
visually evaluating the position of substantially the entire marker relative to the internal tissues as shown in the second composite image on the display.

8. The method of claim 7, wherein the marker comprises at least one chosen from a pin, a drill, an implant, a surgical guide, and a screw.

9. The method of claim 8, wherein said non-radiographic scanning comprises laser scanning.

10. The method of claim 7, wherein said scanning the anatomical portion of the patient to produce the 3-dimensional image including the depiction of internal tissues comprises pre-operative scanning.

11. The method of claim 10, wherein said pre-operative scanning comprises at least one chosen from (i) an X-ray, (ii) a CT scan, (iii) a CBCT scan, and (iv) an MRI scan.

12. The method of claim 7, wherein the marker comprises a fiducial marker on a surgical guide.

13. The method of claim 7, wherein said obtaining a 3-dimensional image of the marker comprises at least one of (i) scanning the marker using a radiographic or non-radiographic imaging device, and (ii) obtaining the 3-dimensional image of the marker from a computer database.

14. A method of evaluating the position of an opening formed in body tissue, said method comprising:
scanning an anatomical portion of a patient to produce an initial 3-dimensional image thereof including a depiction of both internal tissues and exposed surfaces of the anatomical portion;
performing non-radiographic scanning of the exposed surfaces of the anatomical portion of the patient and a proximal end portion of a marker that is positioned in an opening formed in the anatomical portion of the patient, the marker having a distal end portion that is disposed in the opening, and the proximal end portion of the marker projecting outwardly from the opening;
generating a mid-operative 3-dimensional image of the exposed surfaces of the anatomical portion and of exposed surfaces of the proximal end portion of the marker as a result of said non-radiographic scanning;
overlaying and aligning the mid-operative 3-dimensional image of the exposed surfaces of the anatomical portion and the exposed surfaces of the proximal end portion of the marker with the initial 3-dimensional image of the internal tissues and exposed surfaces of the anatomical portion of the patient to produce a first composite image on a display;
obtaining a 3-dimensional image representation of substantially the entire marker including the proximal and distal end portions thereof;
overlaying and aligning the 3-dimensional image representation of substantially the entire marker with the exposed surfaces of the proximal end portion of the marker that appear in the first composite image to create a second composite image on the display; and
visually confirming, via reference to the second composite image on the display, the 3-dimensional position of the distal end portion of the marker relative to the internal tissues of the anatomical portion of the patient.

15. The method according to claim 14, wherein said scanning the anatomical portion of the patient to produce the initial 3-dimensional image thereof comprises performing at least one chosen from (i) an X-ray, (ii) a CT scan, (iii) a CBCT scan, and (iv) an MRI scan.

16. The method according to claim 14, wherein said non-radiographic scanning of the anatomical portion of the patient and the proximal end portion of the marker comprises optical laser scanning.

17. The method according to claim 14, wherein the marker comprises at least one chosen from (i) a pin, (ii) a drill, (iii) a surgical implant, and (iv) a screw.

18. The method according to claim 14, wherein said overlaying and aligning the mid-operative 3-dimensional image with the initial 3-dimensional image comprises aligning at least one fiducial marker that is visible in both the mid-operative 3-dimensional image and the initial 3-dimensional image.

19. The method according to claim 18, wherein the fiducial marker comprises at least one chosen from (i) a tooth, (ii) an exposed portion of bone, and (iii) a portion of a surgical guide that is fitted to the anatomical portion of the patient.

20. The method according to claim 16, wherein the opening formed in the anatomical portion of the patient comprises an osteotomy.

21. The method according to claim 16, wherein said obtaining the 3-dimensional image representation of substantially the entire marker comprises at least one chosen from (i) selecting the 3-dimensional image representation of the marker from an electronic database, (ii) optically scanning the marker to create the 3-dimensional image representation thereof, and (iii) using ionizing radiation to scan the marker and create the 3-dimensional image representation thereof.

22. A method of evaluating the position of a marker relative to anatomical tissue during a medical procedure, said method comprising:
scanning an anatomical portion of a patient to produce a pre-operative 3-dimensional image thereof including a depiction of internal tissues at the anatomical portion;
preparing a 3-dimensional physical model including the outer surfaces of a portion of the patient's anatomy corresponding to the scanned portion;
positioning a marker in a desired location and orientation at the physical model;
scanning the physical model with an electronic image scanning device to produce a 3-dimensional image of the physical model, wherein at least a portion of the marker is captured in the 3-dimensional image of the physical model; and
overlaying and aligning the 3-dimensional image of the physical model and the marker with the pre-operative 3-dimensional image of the anatomical portion of the patient to create a composite image on a display; and
visually verifying, with reference to the composite image, the position and orientation of the marker in the physical model relative to the internal tissues of the corresponding anatomical portion of the patient.

23. The method according to claim 22, further comprising:
after verification, fabricating a surgical guide on the physical model, the surgical guide defining an opening that corresponds to the marker, whereby the surgical guide is configured to align a surgical tool with the location and orientation at the anatomical portion of the patient that substantially corresponds to the location and orientation of the marker at the physical model.

24. The method according to claim 23, wherein said preparing the 3-dimensional physical model comprises at least one chosen from (i) performing an optical scan of the portion of the patient's anatomy without the use of ionizing radiation and creating the physical model from resulting optical scan data using a rapid prototyping apparatus, (ii) performing an optical scan of a molded impression of the patient's anatomy and creating the physical model from resulting optical scan data using a rapid prototyping apparatus, and (iii) using a molded impression of the patient's anatomy and pouring a cast thereof.

25. The method according to claim 23, wherein said scanning the physical model to produce a 3-dimensional image thereof comprises the use of non-ionizing radiation, wherein only an exposed portion of the marker is captured in the 3-dimensional image of the physical model.

26. The method according to claim 25, further comprising:
obtaining a 3-dimensional image of the marker; and
overlaying and aligning the 3-dimensional image of the marker with the exposed portion of the marker in the 3-dimensional image of the physical model.

27. The method according to claim 23, wherein the marker comprises a radiopaque material and said scanning the physical model to produce a 3-dimensional image thereof comprises the use of ionizing radiation, wherein substantially the entirety of the marker is captured in the 3-dimensional image of the physical model.

28. The method according to claim 27, wherein the marker comprises at least one chosen from a dental filling material, barium sulfate acrylic monomer, a pin, a drill, a surgical implant, a surgical guide, and a screw.

29. A method of evaluating the accuracy of a surgical guide for use in creating an opening in anatomical tissue, said method comprising:
scanning an anatomical portion of a patient to produce an initial 3-dimensional image thereof including a depiction of internal tissues at the anatomical portion;
preparing a physical model of the outer surfaces of at least a portion of the anatomical portion of the patient;
placing a surgical guide on the physical model;
creating the opening in the physical model using the surgical guide;
at least partially filling the opening in the physical model with a radiopaque material;
scanning the physical model with an electronic image scanning device to produce a 3-dimensional image of the physical model and the radiopaque material;
overlaying and aligning the 3-dimensional image of the physical model including the radiopaque material with the initial 3-dimensional image of the patient anatomy to create a composite image on a display; and
verifying the position and orientation of the opening created in the physical model relative to the patient's internal tissues in the composite image.

30. The method according to claim 29, wherein said creating the opening in the physical model comprises drilling a hole in the physical model, and wherein the radiopaque material is generally cylindrical in shape.

31. The method according to claim 29, wherein the radiopaque material comprises at least one chosen from a dental filling material, barium sulfate acrylic monomer, a pin, a drill, a surgical implant, a surgical guide, and a screw.

32. A method of producing a surgical guide for use in modifying anatomical tissue, said method comprising:
scanning an anatomical portion of the patient to produce an initial 3-dimensional image thereof including a depiction of internal tissues;
preparing a first physical model of the outer surfaces of at least a portion of the anatomical portion of the patient;
creating an opening in the first physical model with reference to the initial 3-dimensional image as a guide;
at least partially filling the opening in the first physical model with a radiopaque material;
scanning the first physical model to produce a 3-dimensional image thereof, including the radiopaque material;
overlaying and aligning the 3-dimensional image of the first physical model with the initial 3-dimensional image of the patient anatomy to create a composite image on a display;
verifying the position and location of the opening in the first physical model relative to the internal tissues in the anatomical portion of the patient with reference to the composite image;
after verifying the position and location of the opening in the first physical model, inserting a guide element in the opening in the first physical model;
placing a surgical guide blank on the first physical model; and
forming a surgical guide opening in the surgical guide blank based on the position of the guide element relative to the surgical guide blank, to thereby create a surgical guide capable of guiding a surgical tool.

33. The method according to claim 32, further comprising:
preparing a second physical model that is substantially identical to the first physical model;
placing the surgical guide on the second physical model;
creating an opening in the second physical model using the surgical guide;
at least partially filling the opening in the second physical model with a radiopaque material;
scanning the second physical model to produce a 3-dimensional image thereof, including the radiopaque material;
overlaying and aligning the 3-dimensional image of the second physical model with the initial 3-dimensional image of the anatomical portion of the patient to create a second composite image on the display; and
verifying the position and location of the opening in the second physical model relative to the internal tissues in the anatomical portion of the patient with reference to the second composite image.

34. A method of evaluating the position of a marker relative to anatomical tissue in a medical operation, said method comprising:
scanning an anatomical portion of a patient to produce a pre-operative 3-dimensional image thereof, including a depiction of internal tissues at the anatomical portion;
creating a 3-dimensional image representation of the outer surfaces of the anatomical portion of the patient and the distal portion of a marker, from a negative impression of the anatomical portion of the patient in which a distal end portion of a marker was positioned, the negative impression having a proximal end portion of the marker embedded therein;
overlaying and aligning the 3-dimensional image of the representation of the outer surfaces of the anatomical portion of the patient and of the distal portion of the marker, with the pre-operative 3-dimensional image of the patient anatomy, to create a composite image on a display; and
verifying, with reference to the composite image, the position and orientation of an opening in the anatomical portion of the patient relative to the internal tissues, wherein the position and orientation of the opening corresponds to the position and orientation of the marker in the negative impression.

35. The method according to claim 34, wherein said creating the 3-dimensional image representation of the outer surfaces of the anatomical portion of the patient and the distal portion of the marker comprises laser-scanning the negative impression with the marker partially embedded therein.

36. The method according to claim 34, wherein said creating the 3-dimensional image representation of the outer surfaces of the anatomical portion of the patient comprises:
creating a physical model of the anatomical portion of the patient using the negative impression, the physical model either (i) incorporating the marker or (ii) defining an opening left by removal of the marker; and
scanning the physical model of the anatomical portion of the patient with an electronic image scanning device.
